(19) United States
(12) Patent Application Publication
(10) Pub. No.: US 2013/0267681 A1
(43) Pub. Date: Oct. 10, 2013

(54) CHEMICAL LIGATION BY RING OPENING OF OXO-THIOMORPHOLINES

(71) Applicants: Laurence Marius Harwood, Reading (GB); Donald A. Wellings, Runcorn (GB); David John Moody, Falkland (GB)

(72) Inventors: Laurence Marius Harwood, Reading (GB); Donald A. Wellings, Runcorn (GB); David John Moody, Falkland (GB)

(73) Assignee: THE UNIVERSITY OF READING, Reading, Berkshire (GB)

(21) Appl. No.: 13/816,924

(22) PCT Filed: Aug. 12, 2011
(86) PCT No.: PCT/GB11/01212

§ 371 Date: Jun. 26, 2013

(30) Foreign Application Priority Data
Aug. 13, 2010 (GB) 1013666.1

(51) Int. Cl. C07K 1/06; C07K 5/103; C07D 279/12; C07K 5/083

(52) U.S. Cl. CPC C07K 1/063; C07K 5/0806; C07K 5/1008; C07D 279/12
USPC 530/330; 530/331; 544/58.2

(57) ABSTRACT

The invention discloses processes for preparing compounds comprising an α-amino acid motif. The compounds are useful in e.g. the chemical ligation of peptides.

The invention relates to processes for the synthesis of molecules comprising an α-amino acid unit, in particular peptides, and to intermediates useful in the synthesis of such compounds.

### BACKGROUND TO THE INVENTION

Peptides are of central importance in biological systems. In addition, peptides find use in pharmaceutical, agrochemical and other commercial applications.

Chemical synthesis of peptides is a large field of academic endeavour, and many successful approaches to the synthesis of peptides have been developed over the years. The chemical synthesis of peptides allows for the production of the substances on a scale not possible by extraction and purification from natural sources; furthermore, it allows for the incorporation of non-natural amino acids into the peptide structure.

Many chemical syntheses of peptides are linear in approach, linking amino acids in a stepwise fashion. Conventional synthetic techniques start from the C-terminus and form peptide bonds residue by residue, extending in the direction of the N-terminus. This is referred to as C to N synthesis. The problem with such an approach is that even if the individual peptide bond forming reactions are high-yielding, the overall yield rapidly drops off with each extra residue that is added.

The convergent synthesis of peptides presents an attractive alternative to a linear approach. In a convergent synthesis, small peptide fragments are constructed which are subsequently brought together in coupling reactions, referred to as “chemical ligation”.

Native Chemical Ligation is amongst the most useful of these techniques. Native Chemical Ligation allows the combination of two unprotected peptide fragments by utilising the coupling reaction of an α-thioester (I) with a peptide having an N-terminal cysteine (II). The reaction proceeds rapidly and in high yield: a reversible trans-thioesterification reaction gives thioester linked product (III), which subsequently undergoes spontaneous intramolecular rearrangement to give desired peptide product (IV) (Scheme 1).

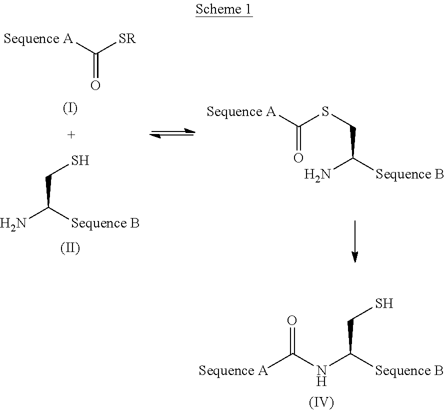

However, Native Chemical Ligation is severely limited in scope. It requires the presence of a cysteine residue at the site of ligation, which may not always be present, or if present, may not allow for the most efficient subdivision of the peptide. A further problem is the synthesis of the peptide thioesters (I); the conditions required for thioester formation are generally incompatible with the FMOC protecting group, that most commonly used in solid phase peptide synthesis.

There remains a need for a technique of chemical ligation which is not dependent on the presence of a cysteine residue at the site of ligation. There remains a need for a technique of chemical ligation which is compatible with the FMOC protecting group.

The present invention addresses these and other problems of the prior art.

### SUMMARY OF THE INVENTION

In a first aspect, the invention provides a process for the preparation of a compound of formula (V)

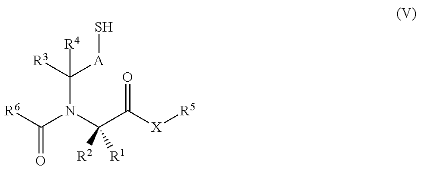

or a salt form thereof,

wherein R¹ to R⁶ are independently selected substituents;

A is selected from a bond, and (CR⁷R⁸)ₙ wherein each of R⁷ and R⁸ is independently selected from the group consisting of H, C₁-C₆ alkyl optionally substituted with from one to five groups independently selected from hydroxy, C₁-C₃ alkoxy, and cyano; C₁-C₆ alkoxycarbonyl, C₁-C₆ haloalkyl, C₆-C₁₀ aryl optionally substituted with from one to five groups independently selected from hydroxy, C₁-C₃ alkoxy, halogen, nitro and cyano; or, taken together with the carbon atom to which they are attached, R⁷ and R⁸ form a C₃-C₇ cycloalkyl ring;

n is 1 or 2;

X is selected from the group consisting of O, S and NR⁹, wherein R⁹ is selected from H and C₁-C₆ alkyl and C₆-C₁₀ aryl

comprising:

i) reacting a compound of formula (VI)

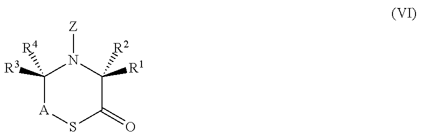

wherein R¹ to R⁴ and A are as defined above and Z is selected from H and a protecting group, with a compound of formula (VII)

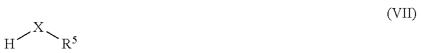

or a reactive derivative thereof, wherein R⁵ and X are as defined above to give a compound of formula (VIII)

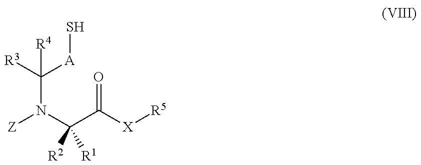

wherein R¹ to R⁵, A and Z are as defined above;

ii) optionally deprotecting compound (VIII) wherein Z is a protecting group to give a compound (VIII) wherein Z is H;

iii) reacting the compound of formula (VIII) wherein Z is H with an acylating agent of formula (IX)

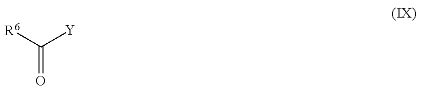

wherein R⁶ is a substituent and Y is a leaving group (which may be inter- or intra-molecular) to give a compound of formula (V).

In a second aspect, the invention provides a compound of the formula (X)

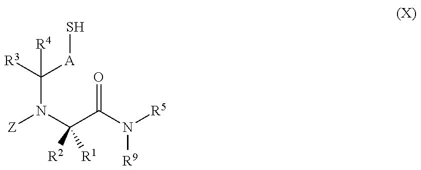

or a salt form thereof, wherein R¹ to R⁵, R⁹, A and Z are as defined above.

In a third aspect, the invention provides a process for the preparation of a compound of formula (XI)

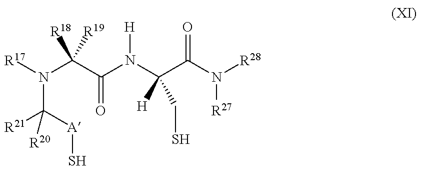

wherein R¹⁷ to R²¹ are independently selected substituents; and

A′ is selected from a bond, and (CR³⁷R³⁸)ₙ wherein each of R³⁷ and R³⁷ is independently selected from the group consisting of H, C₁-C₆ alkyl optionally substituted with from one to three groups independently selected from hydroxy, C₁-C₃ alkoxy, and cyano; C₆-C₁₀ aryl optionally substituted with from one to five groups independently selected from hydroxy, C₁-C₃ alkoxy, halogen, nitro and cyano; C₁-C₆ alkoxycarbonyl, and C₁-C₆ haloalkyl; or, taken together with the carbon atom to which they are attached, R³⁷ and R³⁷ form a C₃-C₇ cycloalkyl ring and n is 1 or 2;

R²⁸ is H or an optionally protected amino acid or peptide;

R²⁷ is H or, in the cases of proline and homoproline, taken together with the nitrogen to which it is attached and the side chain of the adjacent amino acid forms a pyrrolidine or piperidine ring;

comprising

i) reacting a peptide having an N-terminal cysteine residue of formula (XLVI)

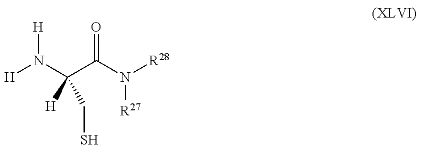

with a compound of formula (XIII)

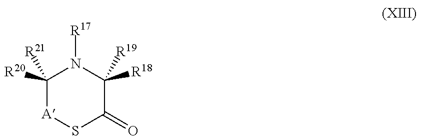

wherein R¹⁷ to R²¹ and A′ are as defined above.

### DETAILED DESCRIPTION

### Preparation of Starting Materials

Compounds of formula (VI) may be prepared by reaction of α-amino acids (XIV) with thiols (XV), and subsequent cyclisation of the thioacid (XVI) (Scheme 2). Suitable reagents for accomplishing these transformations will be apparent to those skilled in the art;

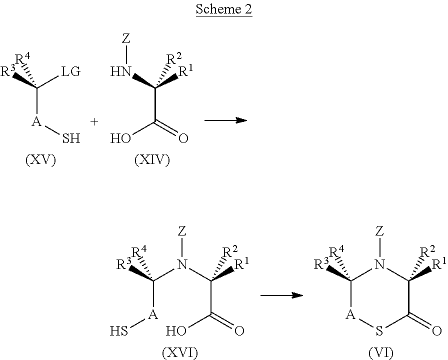

wherein R¹ to R⁴, A and Z have the values ascribed above, and LG represents a leaving group.

In an alternative method, thiocarbonyl compound (XVII) is reacted with aminoacid (XIV) to give thioester (XVIII). Cyclisation of (XVIII) under dehydrating conditions provides either imine (XIX) or enamine (XX; Q represents group A with one substituent replaced by an additional bond to the adjacent carbon atom). Addition of a nucleophile to imine (XIX) (e.g. a Grignard reagent R³MgCl or a hydride equivalent such as NaCNBH₃) gives compound (VI) wherein Z is H. Enamine (XX) may similarly be converted to thiamorpholinone (VI) by known chemistry.

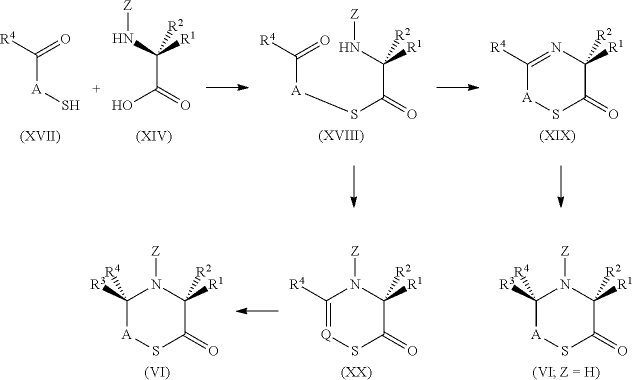

### Preferred Values of Variables

Preferably, A is (CR⁷R⁸)ₙ. Preferably, n is 1. Preferably, R⁷ is H. Preferably, R⁸ is H. More preferably, A is CH₂.

Preferably, R¹ is selected from hydrogen, a C₁-C₁₀ branched or straight chain alkyl group, a mono- or bicyclic heteroaryl group having from 5 to 12 ring members and 1 to 3 heteroatoms independently selected from O, N and S, and a C₆-C₁₂ aryl group; wherein each alkyl, heteroaryl or aryl group is optionally substituted with up to five substituents independently selected from OR¹³, SR¹³, N(R¹³)₂, CO₂R¹³, CON(R¹³)₂, SO₂R¹², SO₃R¹², phenyl, imidazolyl, indolyl, hydroxyphenyl or NR¹³C(═NR¹³)N(R¹³)₂ and each R¹³ is independently selected from hydrogen, C₁-C₆ alkyl and C₆-C₁₂ aryl.

More preferably, R¹ is selected from H, phenyl, and a C₁-C₆ branched or straight chain alkyl group, optionally substituted with phenyl.

Preferably, R² is selected from hydrogen, a C₁-C₁₀ branched or straight chain alkyl group, a mono- or bicyclic heteroaryl group having from 5 to 12 ring members and 1 to 3 heteroatoms independently selected from O, N and S, and a C₆-C₁₂ aryl group; wherein each alkyl, heteroaryl or aryl group is optionally substituted with up to three substituents independently selected from OR¹³, SR¹³, N(R¹³)₂, CO₂R¹³, CON(R¹³)₂, SO₂R¹², SO₃R¹², phenyl, imidazolyl, indolyl, hydroxyphenyl or NR¹³C(═NR¹³)N(R¹³)₂ and each R¹³ is independently selected from hydrogen, C₁-C₆ alkyl and C₆-C₁₂ aryl.

Preferably, at least one of R¹ and R² is hydrogen. More preferably, only one of R¹ and R² is hydrogen.

Preferably, R³ is selected from hydrogen, a C₁-C₁₀ branched or straight chain alkyl group, a mono- or bicyclic heteroaryl group having from 5 to 12 ring members and 1 to 3 heteroatoms independently selected from O, N and S, and a C₆-C₁₂ aryl group; wherein each alkyl, heteroaryl or aryl group is optionally substituted with up to five substituents independently selected from OR¹³, SR¹³, N(R¹³)₂, CO₂R¹³, CON(R¹³)₂, SO₂R¹², SO₃R¹², phenyl, imidazolyl, indolyl, hydroxyphenyl or NR¹³C(═NR¹³)N(R¹³)₂ and each R¹³ is independently selected from hydrogen, C₁-C₆ alkyl and C₆-C₁₂ aryl.

More preferably, R³ is H, or a C₆-C₂ aryl group, optionally substituted as above. More preferably, R³ is a phenyl group, optionally substituted as above. More preferably still, R³ is a phenyl or methoxyphenyl group.

Preferably, R⁴ is selected from hydrogen, a C₁-C₁₀ branched or straight chain alkyl group, a mono- or bicyclic heteroaryl group having from 5 to 12 ring members and 1 to 3 heteroatoms independently selected from O, N and S, and a C₆-C₂ aryl group; wherein each alkyl, heteroaryl or aryl group is optionally substituted with up to five substituents independently selected from OR¹³, SR¹³, N(R¹³)₂, CO₂R¹³, CON(R¹³)₂, SO₂R¹², SO₃R¹², phenyl, imidazolyl, indolyl, hydroxyphenyl or NR¹³C(═NR³)N(R¹³)₂ and each R¹³ is independently selected from hydrogen, C₁-C₆ alkyl, and C₆-C₁₂ aryl. More preferably, R⁴ is a C₆-C₁₂ aryl group, optionally substituted as above. More preferably still, R⁴ is an optionally substituted phenyl group.

Preferably, at least one of R³ and R⁴ is selected from a C₆-C₁₂ aryl group, more preferably an optionally substituted phenyl. More preferably, one of R³ and R⁴ is selected from a C₆-C₂ aryl group, more preferably an optionally substituted phenyl, and the other of R³ and R⁴ is H.

Preferably, X is NR⁹, wherein R⁹ is selected from H, C₁-C₆ alkyl and C₆-C₁₀ aryl and C₁-C₆ alkyl C₆-C₁₀ aryl. More preferably, X is NH.

Preferably, Z is selected from the group consisting of H, benzyl, benzyloxycarbonyl, t-butyloxycarbonyl (BOC), 9H-fluoren-9-ylmethoxycarbonyl (FMOC), allyloxycarbonyl (alloc), and Si((C₁-C₁₀)alkyl). More preferably, Z is H.

Preferably, R⁶ is an optionally protected peptide comprising one or more amino acids, preferably α-amino acids, more preferably naturally occurring amino acids. The optionally protected peptide may be bound to a solid support, for example Merrifield or Wang resin, optionally via a linker.

In an alternative embodiment, at least one of R¹, R², R³, R⁴ and Z, more preferably R³ and R⁴, is attached to a solid support, optionally via a linker. Suitable solid supports and linkers are described in Lloyd-Williams, P.; Albericio, F.; Giralt, E. Chemical Approaches to the Synthesis of Peptides and Proteins; CRC: Boca Raton, Fla., USA, 1997. Suitable solid phase polymers include, but are not limited to, cross-linked polystyrene and polyethylene glycol (PEG) polymers. Suitable linkers include Wang, hydroxymethyl-phenoxy acetyl (HMPA), Rink acid, 2-chlorotrityl chloride, and SASRIN.

A preferred subgroup of compounds (VI) are thiamorpholin-2-ones (XXI), the synthesis of which is described in Synlett 19, 3259-3262, Thieme, 2006, which is incorporated by reference.

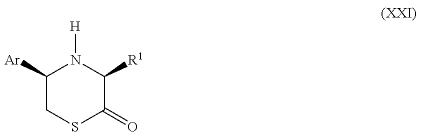

wherein R¹ is selected from methyl, isopropyl, phenyl, benzyl, hydroxybenzyl, indolyl and CH₂CH(CH₃)₂, and Ar is an optionally substituted aryl group, preferably optionally substituted phenyl, most preferably 2,4-dimethoxyphenyl.

In the process of the invention, compound (VI) is reacted with a compound of formula (VII) or a reactive derivative thereof to give compound (VIII) (Scheme 3).

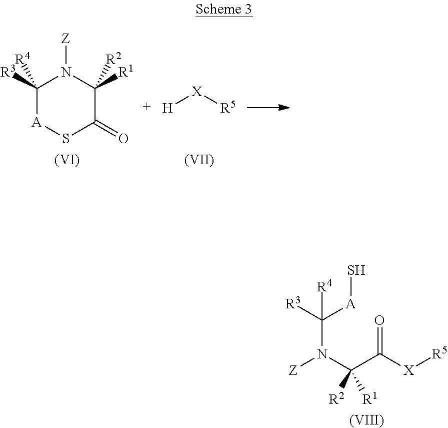

By “reactive derivative thereof” is meant any chemical species capable of participating in the reaction shown in Scheme 3. Preferably, a reactive derivative has the formula (XXII)

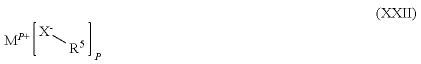

wherein M is selected from a metal (preferably an alkali or alkaline earth metal), or an ammonium cation.

In a very highly preferred embodiment, R⁵ is an optionally protected peptide comprising one or more amino acids, preferably α-amino acids, more preferably naturally occurring amino acids. The optionally protected peptide may be bound to a solid support, for example Merrifield or Wang resin, optionally via a linker. This embodiment is illustrated for a linear peptide having m+1 residues in Scheme 4; the skilled person will of course be aware that the reaction is also possible using branched or cyclic peptides. Compound (VI) is reacted with peptide (XXIII) to give thiol (XXIV)

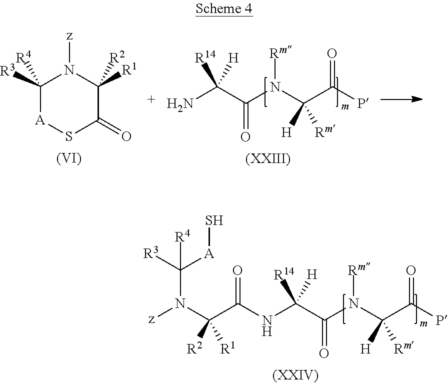

wherein R¹ to R⁴, A and Z are as defined above, R¹⁴ is an amino acid side chain, each R^{m′} is an independently selected amino acid side chain which is optionally protected, or (in the case of proline and homoproline) taken together with R^{m″} represents a group —(CH₂)₃— or —(CH₂)₄—, R^{m″} represents hydrogen, m represents 0 or an integer and P′ represents a protecting group, a solid support or OH.

Suitable conditions for effecting the transformation shown in scheme 3 and scheme 4 will be apparent to the skilled person. Preferably, the reaction is conducted in a solvent. Suitable solvents include ethers (such as diethyl ether, methyl t-butyl ether), haloalkanes (such as dichloromethane), dipolar aprotic solvents (such as dimethylsulfoxide and dimethylformamide) and cyclic solvents (such as morpholine, tetrahydrofuran, dioxane and water).

Catalysts may also be employed. Preferred catalysts are protic acids including mineral acids, for example hydrochloric acid, sulfuric acid, nitric acid, and phosphoric acid, and organic acids such as p-toluenesulfonic acid, trifluoroacetic acid and acetic acid; Lewis acid catalysts such as copper chloride, copper bromide, copper iodide, ammonium iodides, hydrogen iodide, zinc iodide, ferrous iodide, cobaltous iodide, aluminum chloride, trialkyl aluminium compounds (especially boron trifluoride, ferric chloride, zinc chloride, zinc iodide, etc. A preferred class of catalysts are nucleophilic acyl transfer catalysts, including thiols (such as thiophenyl, benzyl mercaptan, 2-mercaptoethanesulfonate, and 4-mercaptophenylacetic acid) and alkylamino pyridines such as dimethylaminopyridine.

Surprisingly, it has been found that the coupling step of (VI) and (VII) to give (VIII) proceeds in good yield, and results in a product which the stereochemistry of the carbon atom marked * is preserved. Epimerisation occurs to a very limited extent, if at all, and to a much lower degree than in prior art methods.

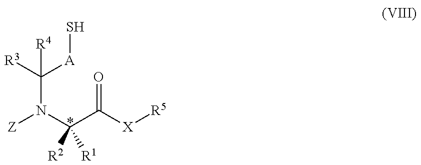

The compounds of formula (VIII) are extremely useful in subsequent synthetic elaborations. In particular, compounds of formula (VIII) wherein Z is H undergo exceptionally facile and high-yielding acylation reactions to give the corresponding amides. This makes compounds of formula (VIII) very valuable in the synthesis of peptides, for example.

In a preferred embodiment, compounds of formula (X) may be obtained in enantiomerically enriched or substantially pure form

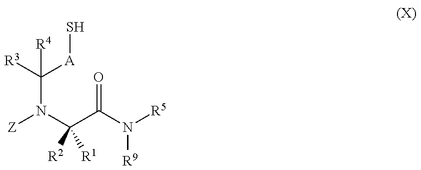

or a salt form thereof, wherein R¹ to R⁵, R⁹ A and Z are as defined above.

In a further preferred embodiment, compounds of formula (V) may be obtained in enantiomerically enriched or substantially pure form

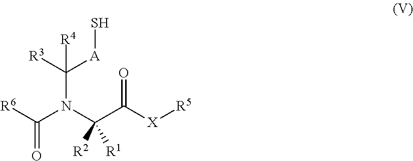

wherein R¹ to R⁶, A and X are as defined above.

Compound (VIII) is deprotected if necessary (i.e. in those embodiments wherein Z is a protecting group) to provide secondary amine (VIII) wherein Z is H (Scheme 5).

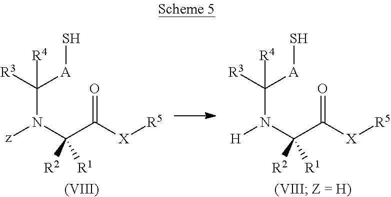

Suitable deprotection conditions will depend on the nature of the group Z, and also the nature of other protecting groups and functionalities present in (VIII). Suitable reagents and conditions are described, for example, in Lloyd-Williams, P.; Albericio, F.; Giralt, E. Chemical Approaches to the Synthesis of Peptides and Proteins; CRC: Boca Raton, Fla., USA, 1997.

In further step (iii) of the process according to the invention, secondary amine (VIII; Z═H) is reacted with acylating agent (IX) (Scheme 6) to give amide (V).

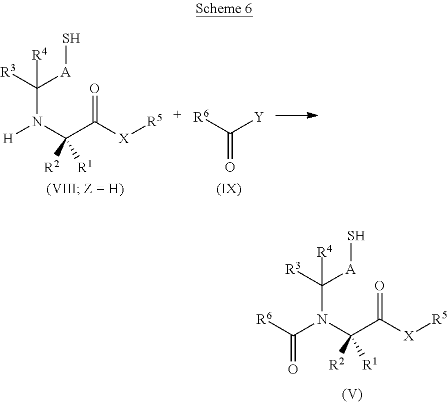

Suitable leaving groups Y include halides (especially fluoride), azides, active esters (such as pentafluorophenyl and oxybenzotriazolyl) and anhydrides. Group Y may also be formed from corresponding carboxylic acid by reaction with any of the known peptide coupling agents known in the art, for example carbodiimides, phosphonium agents and uronium agents. Suitable conditions are set out for example in Lloyd-Williams, P.; Albericio, F.; Giralt, E. Chemical Approaches to the Synthesis of Peptides and Proteins; CRC: Boca Raton, Fla., USA, 1997.

Preferably, compound (IX) is a thioester. Very preferably, Y is a group —SR¹⁵, wherein R¹⁵ is a substituent. Preferably, R¹⁵ is selected from C₁-C₁₀ alkyl, C₆-C₁₀ aryl optionally substituted with from 1 to 3 substituents independently selected from halogen, C₁-C₆ alkyl, C₁-C₆ alkoxy, C₁-C₆ haloalkyl, C₁-C₆ haloalkoxy, nitro and cyano; a mono- or bicyclic heteroaryl group having from 5 to 12 ring members and 1 to 3 heteroatoms independently selected from O, N and S, optionally substituted with from 1 to 3 substituents independently selected from halogen, C₁-C₆ alkyl, C₁-C₆ alkoxy, C₁-C₆ haloalkyl, C₁-C₆ haloalkoxy, nitro and cyano, or (C₁-C₁₀)alkyl(C₆-C₁₀)aryl. Most preferably, Y is SMe.

Surprisingly, it is found that when Y is a group —SR¹⁵, and in particular a group —SMe, the coupling of amine (VIII; Z═H) with acylating agent (IX) proceeds rapidly, in high yield, and with excellent retention of stereochemistry in all stereocentres in the molecule (V). Furthermore, the transformation may be accomplished wherein R⁵ and R⁶ are both peptide groups. This is even the case wherein R⁵ and/or R⁶ are unprotected peptide groups.

Without wishing to be bound by any theory, it is believed that an initial reversible transthioesterification reaction between (VIII; Z═H) and thioester (XXV) gives (XXVI), which rearranges to give thermodynamically more stable amide (V) (Scheme 7).

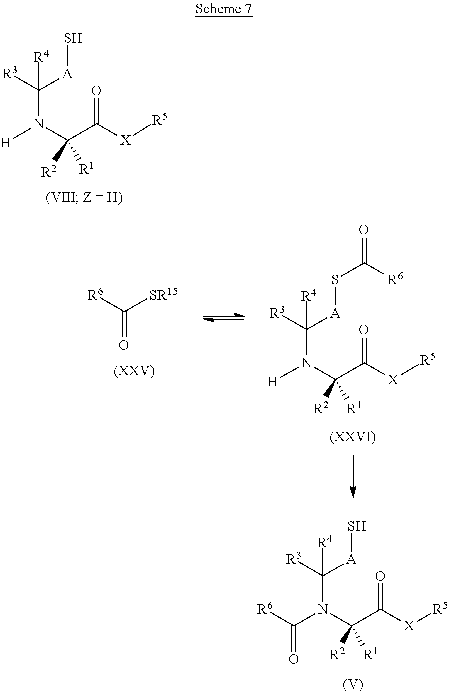

Leaving group Y may be an intramolecular leaving group, for example, covalently attached to the remainder of the molecule by a connecting group M′ (compound XXVII).

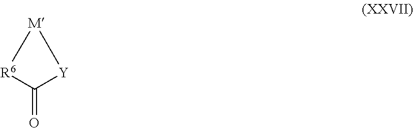

Examples of acylating agents having intramolecular leaving groups include those compounds having β-lactam (XXVIII), aziridinone (XXIX) and α-lactone (XXX) moieties.

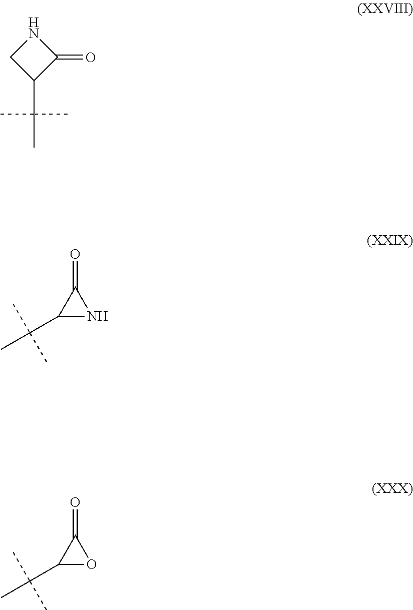

An alternative, preferred class of acylating agent, having an intramolecular leaving group, is cyclic thioester (XIII)

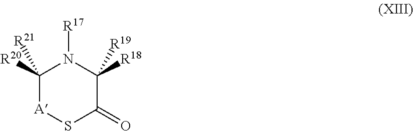

wherein R¹⁷ to R²¹ are independently selected substituents; and A′ is selected from a bond, and (CR³⁷R³⁸)ₙ wherein each of R³⁷ and R³⁸ is independently selected from the group consisting of H, C₁-C₆ alkyl optionally substituted with from one to three groups independently selected from hydroxy, C₁-C₃ alkoxy, and cyano; C₆-C₁₀ aryl optionally substituted with from one to five groups independently selected from hydroxy, C₁-C₃ alkoxy, halogen, nitro and cyano; C₁-C₆ alkoxycarbonyl, and C₁-C₆ haloalkyl; or, taken together with the carbon atom to which they are attached, R³⁷ and R³⁸ form a C₃-C₇ cycloalkyl ring; and

n is 1 or 2.

Although R⁶ may be any substituent, in a particularly preferred embodiment, R⁶ is an optionally protected peptide comprising one or more amino acids, preferably α-amino acids, more preferably naturally occurring amino acids. This embodiment is illustrated for a linear peptide having q+1 residues (Scheme 8). Peptide (XXXII) is coupled with amine (VIII; Z═H) to give amide (XXXIII).

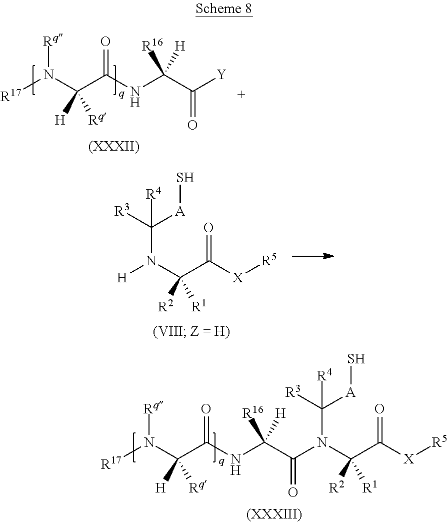

wherein R¹ to R⁴, A and X are as defined above, R¹⁶ is an amino acid side chain, each R^{q′} is an independently selected amino acid side chain which is optionally protected, or (in the case of proline) taken together with R^{4″} represents a group —(CH₂)₃—, R^{q″} represents hydrogen, q represents 0 or an integer and R¹⁷ is selected from H and a protecting group.

In a particularly preferred embodiment, both R⁵ and R⁶ are optionally protected peptides. This embodiment provides an expedient method of linking two shorter peptide fragments. Unlike native chemical ligation, the presence of a cysteine residue is not required.

In a preferred embodiment, compound (V) is converted in a further step to secondary amide (XXXIV) (scheme 9).

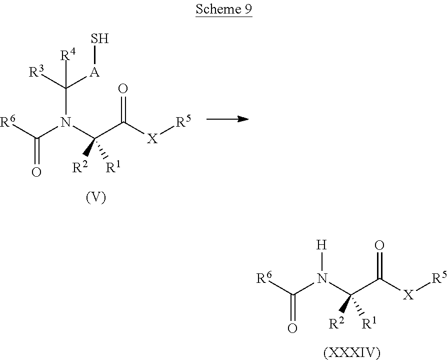

Various methods may be used for achieving the transformation of (V) to (XXXIV). This are known in the art, and will depend on the nature of groups R³, R⁴ and A. In those embodiments in which at least one of R³ and R⁴ is aryl, a preferred method is by Birch reduction (e.g. with lithium in liquid ammonia).

An alternative, preferred method of achieving this transformation, in those embodiments wherein at least one of R³ and R⁴ is aryl substituted by an alkoxy group, is treatment with trifluoroacetic acid.

Further transformations of compound (XXXIV) may be necessary to furnish the final desired product. For example, in embodiments wherein R⁵ and/or R⁶ are protected peptides, deprotection will be preferred. Suitable methods for achieving deprotection will be well known to the peptide chemist.

A further embodiment of the invention provides a process for the preparation of a compound of formula (XXXV) comprising reacting a compound of formula (VIII; Z═H)) with a compound of formula (XIII) (Scheme 10)

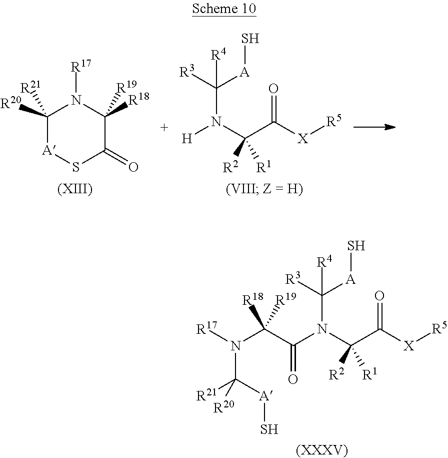

wherein R¹ to R⁵, A and X are as defined above, and wherein R¹⁷ to R²¹ are independently selected substituents; and A′ is selected from a bond, and (CR³⁷R³⁸)ₙ wherein each of R³⁷ and R³⁸ is independently selected from the group consisting of H, C₁-C₆ alkyl optionally substituted with from one to five groups independently selected from hydroxy, C₁-C₃ alkoxy, and cyano; C₆-C₁₀ aryl optionally substituted with from one to five groups independently selected from hydroxy, C₁-C₃ alkoxy, halogen, nitro and cyano; C₁-C₆ alkoxycarbonyl, and C₁-C₆ haloalkyl; or, taken together with the carbon atom to which they are attached, form a C₃-C₇ cycloalkyl ring; and

n is 1 or 2.

Preferably, R¹⁶ is selected from hydrogen, a C₁-C₁₀ branched or straight chain alkyl group, a mono- or bicyclic heteroaryl group having from 5 to 12 ring members and 1 to 3 heteroatoms independently selected from O, N and S, and a C₆-C₁₂ aryl group; wherein each alkyl, heteroaryl or aryl group is optionally substituted with up to three substituents independently selected from OR¹³, SR¹³, N(R¹³)₂, CO₂R³, CON(R¹³)₂, SO₂R¹², SO₃R¹², phenyl, imidazolyl, indolyl, hydroxyphenyl or NR¹³C(═NR¹³)N(R¹³)₂ and each R¹³ is independently selected from hydrogen, C₁-C₆ alkyl and C₆-C₁₂ aryl.

More preferably, R¹⁸ is selected from H and a side chain of a naturally occurring amino acid.

Preferably, R¹⁹ is selected from hydrogen, a C₁-C₁₀ branched or straight chain alkyl group, a mono- or bicyclic heteroaryl group having from 5 to 12 ring members and 1 to 3 heteroatoms independently selected from O, N and S, and a C₆-C₁₂ aryl group; wherein each alkyl, heteroaryl or aryl group is optionally substituted with up to three substituents independently selected from OR¹³, SR¹³, N(R¹³)₂, CO₂R¹³, CON(R¹³)₂, SO₂R¹², SO₃R¹², phenyl, imidazolyl, indolyl, hydroxyphenyl or NR¹³C(═NR¹³)N(R¹³)₂ and each R¹³ is independently selected from hydrogen, C₁-C₆ alkyl and C₆-C₁₂ aryl.

More preferably, R¹⁹ is selected from H and a side chain of a naturally occurring amino acid.

Preferably, at least one of R¹⁸ and R¹⁹ is hydrogen. More preferably, only one of R¹⁸ and R¹⁹ is hydrogen. An advantage of the process of the present invention is that it permits access to both the naturally-occurring (L) forms and synthetic (D) forms of amino acids, i.e. those instances wherein one of R¹⁹ or R¹⁸ is H.

Preferably, R²⁰ is selected from hydrogen, a C₁-C₁₀ branched or straight chain alkyl group, a mono- or bicyclic heteroaryl group having from 5 to 12 ring members and 1 to 3 heteroatoms independently selected from O, N and S, and a C₆-C₁₂ aryl group; wherein each alkyl, heteroaryl or aryl group is optionally substituted with up to three substituents independently selected from OR¹³, SR¹³, N(R¹³)₂, CO₂R¹³, CON(R¹³)₂, SO₂R¹², SO₃R¹², phenyl, imidazolyl, indolyl, hydroxyphenyl or NR¹³C(═NR¹³)N(R¹³)₂ and each R¹³ is independently selected from hydrogen, C₁-C₆ alkyl and C₆-C₁₂ aryl.

Preferably, R²¹ is selected from hydrogen, a C₁-C₁₀ branched or straight chain alkyl group, a mono- or bicyclic heteroaryl group having from 5 to 12 ring members and 1 to 3 heteroatoms independently selected from O, N and S, and a C₆-C₁₂ aryl group; wherein each alkyl, heteroaryl or aryl group is optionally substituted with up to three substituents independently selected from OR¹³, SR¹³, N(R¹³)₂, CO₂R³, CON(R¹³)₂, SO₂R¹², SO₃R¹², phenyl, imidazolyl, indolyl, hydroxyphenyl or NR¹³C(═NR¹³)N(R¹³)₂ and each R¹³ is independently selected from hydrogen, C₁-C₆ alkyl and C₆-C₁₂ aryl.

Preferably, at least one of R²⁰ and R²¹ is selected from a C₆-C₁₂ aryl group, more preferably an optionally substituted phenyl. Preferably, the phenyl group is substituted by from 1 to 3 substituents independently selected from C₁-C₆ alkoxy, preferably methoxy. Most preferably, at least one of R²⁰ and R²¹ is 2,4-dimethoxyphenyl. More preferably, one of R²⁰ and R²¹ is selected from a C₆-C₁₂ aryl group, more preferably an optionally substituted phenyl as defined above, and the other of R²⁰ and R²¹ is H.

Preferably, R¹⁷ is an optionally protected peptide comprising one or more amino acids, preferably α-amino acids, more preferably naturally occurring amino acids. The optionally protected peptide may be bound to a solid support, for example Merrifield or Wang resin, optionally via a linker. This embodiment is illustrated for a linear peptide having r+1 residues (Scheme 11). Cyclic thioester (XXXVI) is reacted with secondary amine (VIII; Z═H) to provide corresponding amide (XXXVII).

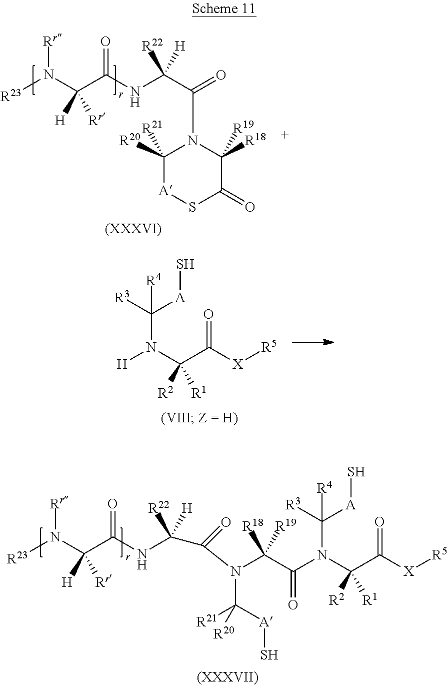

wherein R¹ to R⁵, A and X are as defined above, R²² is an amino acid side chain, each R^{r′} is an independently selected amino acid side chain which is optionally protected, or (in the case of proline) taken together with R^{r′} represents a group —(CH₂)₃—, R^{r″} represents hydrogen, r represents 0 or an integer and R²³ is selected from H, a protecting group and a solid support. Preferably, R²³ is a 9H-fluoren-9-ylmethoxycarbonyl (FMOC) group.

In this aspect, the invention presents compounds (XXXVI) as an alternative to the use of the thioesters (XXV) as shown above in Scheme 7 as coupling partners with compounds (VIII; Z═H). This is advantageous, as the formation of compounds (XXVII) can be accomplished when FMOC protecting groups are present in group R¹⁷, whereas formation of thioesters (XXV) when FMOC protecting groups are present is problematic.

In a very highly preferred embodiment, R¹⁷ is an optionally protected peptide comprising one or more amino acids, preferably α-amino acids, more preferably naturally occurring amino acids, X is NR⁹, wherein R⁹ is selected from H and C₁-C₆ alkyl, and R⁵ is an optionally protected peptide comprising one or more amino acids, preferably α-amino acids, more preferably naturally occurring amino acids.

Compounds of formula (XXXVI) are suitably prepared from peptide (XXXVIII) and thiamorpholinone (XXXIX) using peptide coupling methods known in the art (Scheme 12)

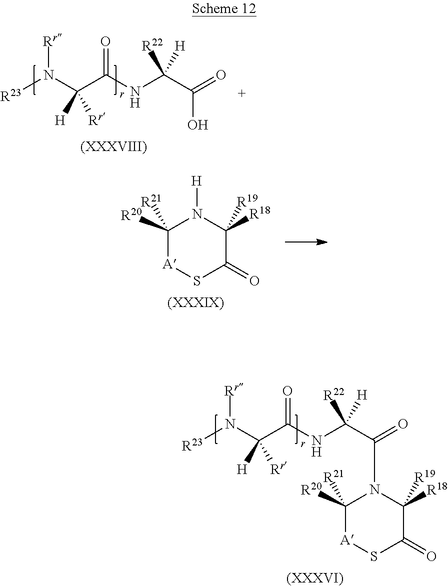

wherein R¹⁸ to R²³, A′, R^{r′}, R^{r″} and r are as defined above. Suitable techniques are given, for example, in WO2007031698.

An alternative method for the preparation of the compounds of formula (XXXVI) is from compounds of formula (XXXIX) in which amino acids may be added in a stepwise fashion using known peptide coupling techniques and conditions. For example, in those embodiments wherein r=0, scheme 12 involves the addition of a single amino acid residue.

An alternative method for the preparation of the compounds of formula (XXXVI) is shown in Scheme 13. Thiamorpholinone (XXXIX) may be ring opened with e.g. methanethiol to give thioester/thiol compound (XXXX). Reaction with thioester (XL) (thioester derivative of peptide (XXXVIII)) gives (after thioester exchange, S to N migration and re-cyclisation with elimination of methanethiol) compound (XXXVI).

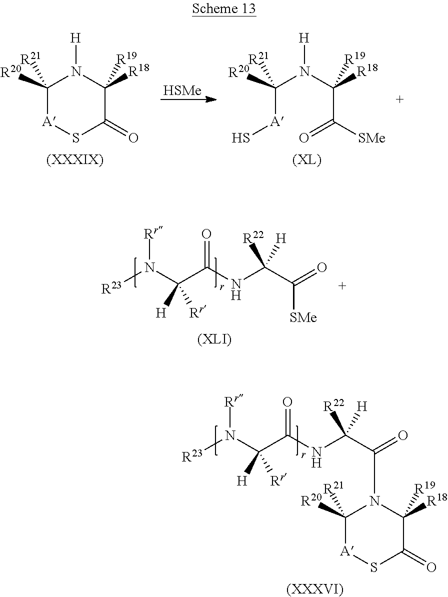

Further amino acids may be added in a stepwise fashion using known peptide coupling techniques and conditions (Scheme 13). N-terminal peptide (XLII) is coupled with amino acid (XLIII) to give chain-extended peptide (XLIV),

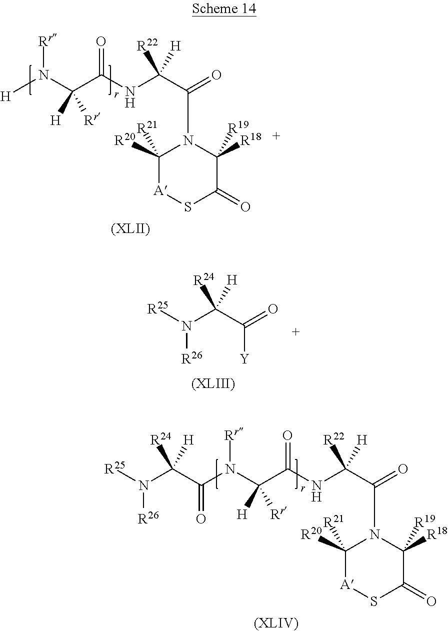

wherein R¹⁸ to R²³, A′, R^{r′}, R^{r″} and r are as defined above, Y is a leaving group or OH, R²⁴ is an amino acid side chain or tin the case of proline) taken together with R²⁵ forms a group —(CH₂)₃—, R²⁵ is H or taken together with R²⁴ forms a group —(CH₂)₃—, and R²⁶ is H or a protecting group. Preferably, R²⁶ is a 9H-fluoren-9-ylmethoxycarbonyl (FMOC) group.

Product (XXXV) may be deprotected to give (XLV) (Scheme 15)

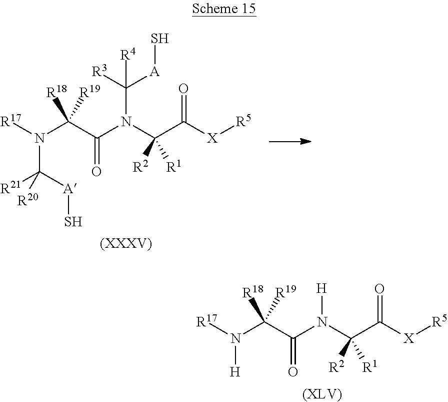

wherein R¹ to R⁵, R¹⁷ to R²¹, A, A′ and X are as defined above. Various methods may be used for achieving the transformation of (XXXIII) to (XL). This are known in the art, and will depend on the nature of groups R³, R⁴, R²⁰, R²¹, A and A′. In those embodiments in which at least one of R³, R⁴, R²⁰ and R²¹ is aryl, a preferred method is by Birch reduction (e.g. with lithium in liquid ammonia).

An alternative, preferred method of achieving this transformation, in those embodiments wherein at least one in which at least one of R³, R⁴, R²⁰ and R²¹ is aryl substituted by an alkoxy group, is treatment with trifluoroacetic acid.

Further transformations of compound (XLV) may be necessary to furnish the final desired product. For example, in embodiments wherein R⁵ and/or R¹⁷ are protected peptides, deprotection will be preferred.

A further aspect, the invention provides an alternative to the use of thioesters in native chemical ligation. Compound (XIII) reacts with N-terminal cysteine peptide (XLVI) to give coupled product (XLVII) (Scheme 15)

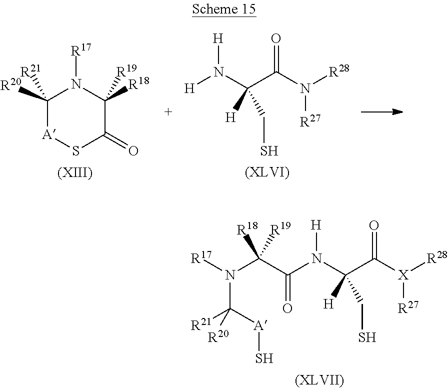

wherein R¹⁷ to R²¹, and A′ are as defined above, R²⁸ is H or is an optionally protected peptide comprising one or more amino acids, preferably α-amino acids, more preferably naturally occurring amino acids, R²⁷ is H or taken together with the nitrogen to which it is attached and the side chain of the adjacent amino acid forms a pyrrolidine ring (e.g. in the case of proline).

Peptide (XLVII) may be deprotected to give (XLVIII) (Scheme 16)

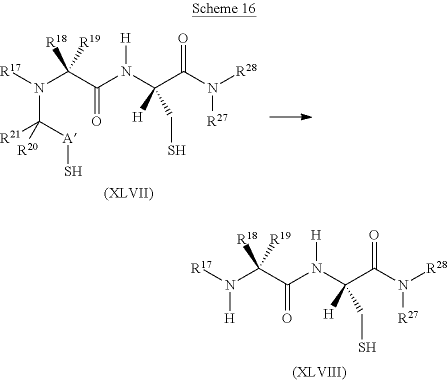

wherein R¹⁷ to R²⁰, R²⁷, R²⁸ and A′ are as defined above. Suitable conditions are as set out above.

### Substituent

“Substituent” is used in the sense that will be readily understood by the person skilled in the art as an atom or group of atoms covalently linked to the remainder of the molecule in question, and may include polymeric, anionic and cationic groups. The term includes hydrogen.

### Alkyl

Alkyl, as used herein refers to an aliphatic hydrocarbon chain and includes straight and branched chains e.g. of 1 to 10, preferably 1 to 6 carbon atoms such as methyl, ethyl, n-propyl, isopropyl, n-butyl, isobutyl, sec-butyl, t-butyl, n-pentyl, isopentyl, neo-pentyl, n-hexyl, and isohexyl.

### Alkoxy

Alkoxy as used herein refers to the group —O-alkyl, wherein alkyl is as defined above. Examples of alkoxy groups include methoxy, ethoxy, n-propoxy, isopropoxy, n-butoxy, isobutoxy, sec-butoxy, t-butoxy, n-pentoxy, isopentoxy, neo-pentoxy, n-hexyloxy, and isohexyloxy.

### Halogen

Halogen, halide and halo refer to iodine, bromine, chlorine and fluorine.

Aryl As used herein, “aryl” refers to an unsaturated aromatic carbocyclic group of from 6 to 10 carbon atoms having a single ring (e.g., phenyl) or multiple condensed (fused) rings, at least one of which is aromatic (e.g., indanyl, naphthyl). Preferred aryl groups include phenyl, naphthyl and the like.

### Heteroaryl

The term “heteroaryl” refers to a ring system containing 5 to 12 ring atoms, at least one ring heteroatom and consisting either of a single aromatic ring or of two or more fused rings, at least one of which is aromatic. Ring systems contain up to three heteroatoms which will preferably be chosen independently from nitrogen, oxygen and sulfur. Examples of such groups include pyridyl, pyridazinyl, pyrimidinyl, pyrazinyl, triazinyl, furanyl, thiophenyl, oxazolyl, isoxazolyl, oxadiazolyl, thiazolyl, isothiazolyl, thiadiazolyl, pyrrolyl, pyrazolyl, imidazolyl, triazolyl and tetrazolyl. Examples of bicyclic groups are benzothiophenyl, benzimidazolyl, benzothiadiazolyl, quinolinyl, cinnolinyl, quinoxalinyl and pyrazolo[1,5-a]pyrimidinyl.

### Peptide

As used herein, “peptide” refers to at least two covalently attached amino acids, which includes polypeptides, and oligopeptides. The peptide may be made up of naturally occurring amino acids and peptide bonds, or synthetic peptidomimetic structures. Thus, “amino acid” or “peptide residue” as used herein means both naturally occurring and synthetic amino acids. For example, homo-phenylalanine, citrulline, and norleucine are considered amino acids for the purposes of the invention. “Amino acids” also includes imino residues such as proline and hydroxyproline. The side chains may be either the D- or L-configuration, or combinations thereof. Thus, the peptides may have one or more D-isomer amino acids, up to all of the amino acids of the peptide being the D-isomer. Although the bond between each amino acid is typically an amide or peptide bond, it is to be understood that peptide also includes analogs of peptides in which one or more peptide linkages are replaced with other than an amide or peptide linkage, such as a substituted amide linkage, an isostere of an amide linkage, or a peptide or amide mimetic linkage (see, e.g., Spatola, “Peptide Backbone Modifications,” in Chemistry and Biochemistry of Amino Acids Peptides and Proteins, Weinstein, ed., Marcel Dekker, New York (1983); Olson, G. L. et al, J. Med. Chem. 36:3039-3049 (1993); and Ripka and Rich, Curr. Opin. Chem. Biol. 2:441-452 (1998)). The term “peptide” encompasses peptides of natural origin, those synthetically derived, and those of semi-synthetic origin.

### Optional Substitution

“Optionally substituted” as used herein means the group referred to can be substituted at one or more positions by any one or any combination of the radicals listed thereafter.

### Protecting Group

As used herein, “protecting group” refers to a group that is joined to a reactive group (e.g., a hydroxyl or an amine) on a molecule. The protecting group is chosen to prevent reaction of the particular radical during one or more steps of a chemical reaction. Generally the particular protecting group is chosen so as to permit removal at a later time to restore the reactive group without altering other reactive groups present in the molecule. The choice of a protecting group is a function of the particular radical to be protected and the compounds to which it will be exposed. The selection of protecting groups is well known to those of skill in the art. See, for example Greene et al., Protective Groups in Organic Synthesis, 2nd ed., John Wiley & Sons, Inc. Somerset, N.J. (1991), which is incorporated by reference herein in its entirety. The term “protection” refers to the introduction of such a group, and the term “deprotection” to its removal. The term “protected” refers to a molecule comprising such a group.

### Leaving Group

As used herein, “leaving group” refers to any group that can be replaced by a nucleophile upon nucleophilic substitution. Example leaving groups include, halo (F, Cl, Br, I), hydroxyl, alkoxy, mercapto, thioalkoxy, triflate, alkylsulfonyl, substituted alkylsulfonate, arylsulfonate, substituted arylsulfonate, heterocyclosulfonate or trichloroacetimidate. Representative examples include p-(2,4-dinitroanilino)benzenesulfonate, benzenesulfonate, methylsulfonate, p-methylbenzenesulfonate, p-bromobenzenesulfonate, trichloroacetimidate, acyloxy, 2,2,2-trifluoroethanesulfonate, imidazolesulfonyl and 2,4,6-trichlorophenyl.

### Labelled Compounds

The methods of the invention may be used in the preparation of labelled compounds, such as compounds comprising deuterium, tritium and carbon-13.

### EXAMPLES

General—mass spectra were collected using electrospray ionization.

### Example 1

### Preparation of H-AlaAlaAla-OH (XLVI)

### Step 1

syn-3S-methyl-5R-(2,4-dimethoxy)phenylthiamorpholinone (XLIX) is reacted with L-alanine ^{t}butyl ester (L) to obtain (LI).

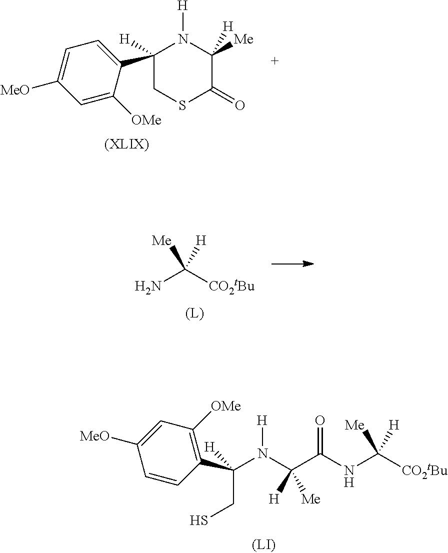 [SMILES: COc1ccc([C@@H]2CSC(=O)[C@H](C)N2)c(OC)c1 ; C[C@H](N)C(=O)OC(C)(C)C ; COc1ccc([C@H](CS)N[C@@H](C)C(=O)N[C@@H](C)C(=O)OC(C)(C)C)c(OC)c1]

### Step 2

(LI) is reacted with N-fmoc-L-alanine S methyl ester (LII) to give compound (LIII).

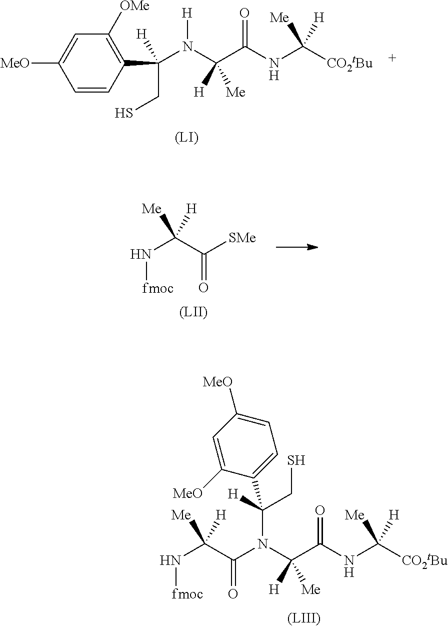

### Step 3

(LIII) is subjected to treatment with i) trifluoroacetic acid and ii) piperidine to give ala-ala-ala (compound (LV)).

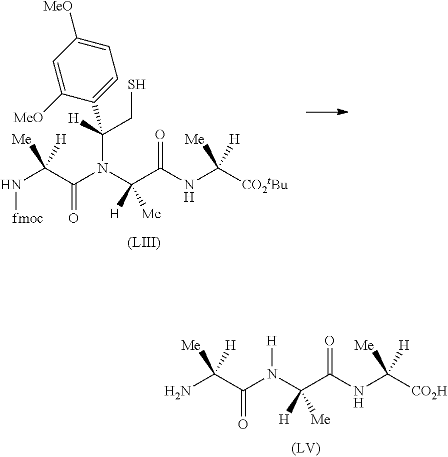

### Example 2

### Preparation of H-AlaAlaAlaAla-OH (LVII)

### Step 1

syn-3S-methyl-5R-(2,4-dimethoxy)phenylthiamorpholinone (XLIX) is reacted with N-FMOC L-alanine acid chloride (LVIII) in the presence of base to obtain (LIX).

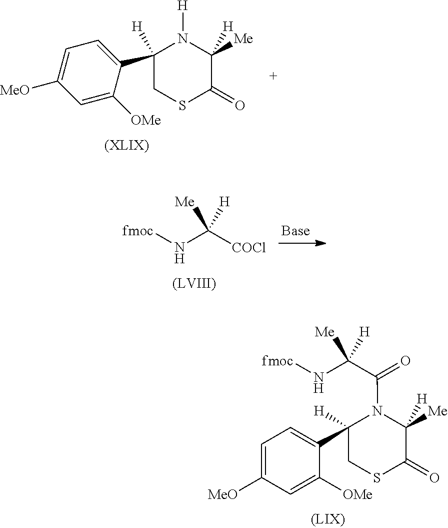

### Step 2

The product of Step I (LIX) is reacted with (LI) from Example 1 to give coupled product (LX)

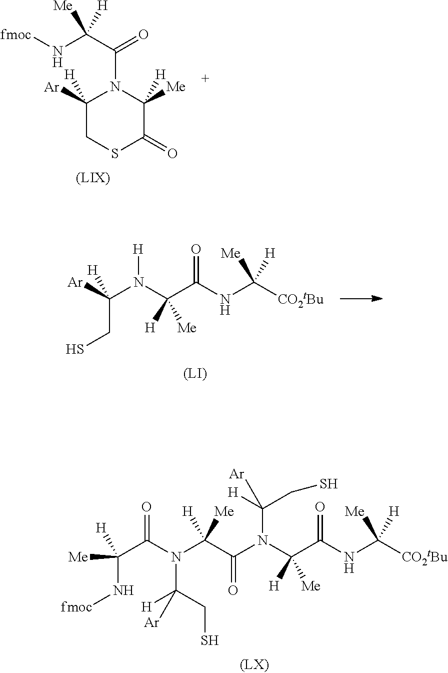

wherein Ar=2,4 dimethoxyphenyl

### Step 3

(LX) is subjected to treatment with i) trifluoroacetic acid and ii) piperidine to give compound (LXI).

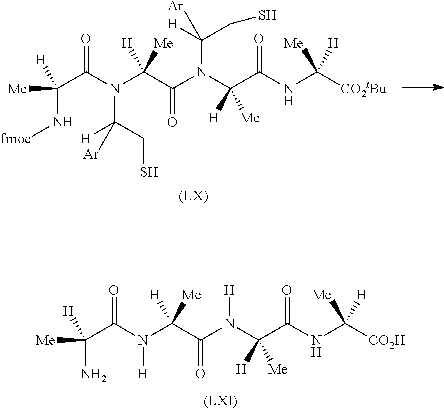

### Example 3

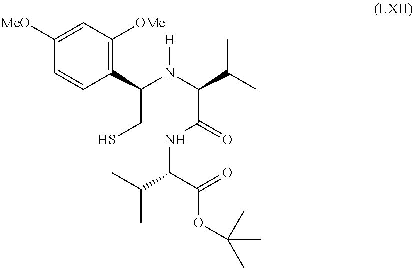 [SMILES: COc1ccc([C@H](CS)N[C@H](C(=O)N[C@H](C(=O)OC(C)(C)C)C(C)C)C(C)C)c(OC)c1]

To a solution of (3S,5R)-5-(2,4-dimethoxyphenyl)-3-isopropylthiomorpholin-2-one (200 mg, 1 equiv) in DMSO D6 (1 mL), was added t-butyl valine hydrochloride (690 mg, 5 equiv) and triethylamine (330 μL, 5 equiv), the solution was then stirred overnight at 4000. The solution then filtered through a short silica G pad, eluting with 3:1 ether:tetrahydrofuran. The filtrate was then concentrated in vacuo to give (LXII) as a yellow oil to give 276 mg.

### Example 4

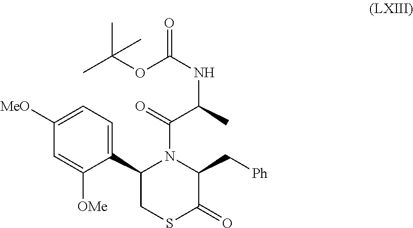 [SMILES: COc1ccc([C@@H]2CSC(=O)[C@H](Cc3ccccc3)N2C(=O)[C@H](C)NC(=O)OC(C)(C)C)c(OC)c1]

To a solution of (3S,5R)-3-benzyl-5-(2,4-dimethoxyphenyl)thiomorpholin-2-one (100 mg, 1 equiv) in anhydrous tetrahydrofuran (2 mL) was added Boc-alanine-OH (70 mg, 1 equiv). HBTU (115 mg, 1 equiv) was then added followed by diisopropylethylamine (80 μl, 4 equiv) and HOBt (40 mg, 1 equiv,) The mixture was then stirred for two hours and concentrated in vacuo to give (LXIII) 40 mg.

### Example 5

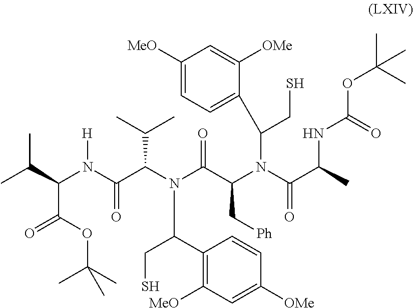 [SMILES: COc1ccc(C(CS)N(C(=O)[C@H](Cc2ccccc2)N(C(=O)[C@H](C)NC(=O)OC(C)(C)C)C(CS)c2ccc(OC)cc2OC)[C@H](C(=O)N[C@@H](C(=O)OC(C)(C)C)C(C)C)C(C)C)c(OC)c1]

To a solution of compound (LXII) (20 mg, 1 equiv) in anhydrous tetrahydrofuran (1 mL) compound (LXIII) was added (19 mg, 1 equiv), the reaction was then stirred overnight, and then concentrated in vacuo to give (LXIV) as a crude as a colourless solid (38 mg) (m/z=983.5224 (MH⁺); calculated for C₅₁H₇₄N4O₁₁S₂ 982.4795)

### Example 6

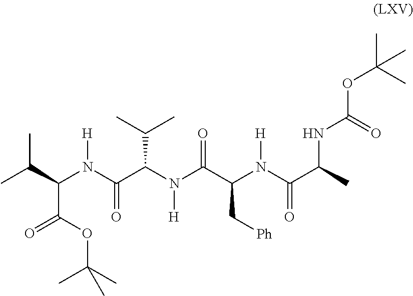 [SMILES: CC(C)[C@H](NC(=O)[C@H](Cc1ccccc1)NC(=O)[C@H](C)NC(=O)OC(C)(C)C)C(=O)N[C@@H](C(=O)OC(C)(C)C)C(C)C]

Compound (LXIV) was dissolved in 1 mL of a solution composed of 95% trifluoroacetic acid, 2.5% water and 2.5% triethylsilane (v/v), and stirred overnight. The reaction was then concentrated in vacuo, diethyl ether was added to the residue which was triturated. The solution was decanted but retained. The insoluble residue product was then dried in vacuo at 3500 to give (LXV) (MS m/z=468).

### Example 7

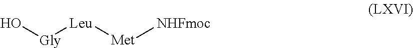

Peptide fragment (LXVI) was prepared on an Applied Bio systems 430A peptide synthesizer using standard 0.25M FastMoc chemistry program. The resin was then cooled on ice to which the 1.5 mL of the deprotection solution (0.75 g crystalline phenol, 0.25 mL EDT, 0.5 mL thioanisole, 0.5 mL water, dissolved in 10 mL TFA) was added. The solution was then warmed to room temperature and stirred for 1.5 hours. The mixture was filtered through a fine pore sinter, the flask was then washed with TFA (1 mL); these rinsings were also filtered. The flask was finally washed with DCM (10 mL) which was combined with the TFA filtrate. The solution was concentrated in vacuo to 1 mL, the solution was then diluted with water (10 mL) and transferred to a separating funnel. The aqueous mixture was then extracted with diethyl ether (3×10 mL), The aqueous layer was then freeze dried to give a solid (204 mg) (compound LXVI).

### Example 8

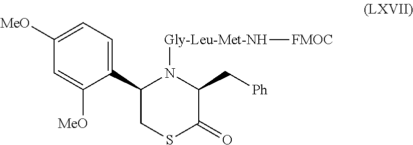

(3S,5R)-3-benzyl-5-(2,4-dimethoxyphenyl)thiomorpholin-2-one (340 mg, 2 equiv) and (LXVI) (135 mg, 1 equiv) were dissolved in NMP (1 mL) to which HBTU (152 mg, 1 equiv), DIPEA (260 μl, 4 equiv) and HOBt (66 mg, 1 equiv) were added. The mixture was allowed to stand overnight. The mixture (containing product (LXVII)) was then taken crude to the next stage.

### Example 9

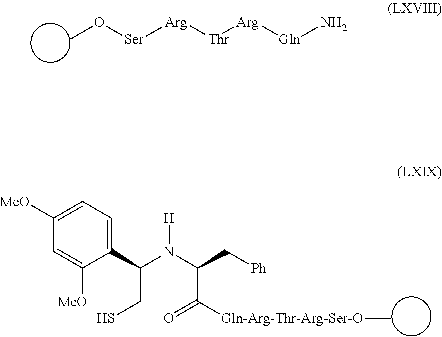

Peptide fragment (LXVIII) was prepared on an Applied Bio systems 430A peptide synthesizer using the standard 0.25M FastMoc chemistry program. Compound (LXVIII) (80 mg of peptide, 1 equiv) was added to NMP (1 mL), then (3S,5R)-3-benzyl-5-(2,4-dimethoxyphenyl)thiomorpholin-2-one (215 mg, 5 equiv) and triethylamine (63 μl, 5 equiv) were added. The solution was stirred overnight, and the resulting resin (compound LXIX) was then washed with NMP by filtration. Theoretical yield 122 mg.

### Example 10

### HO-Ser-Arg-Thr-Arg-Gln-Phe-Phe-Gly-Leu-Met-NH₂ LXX

An equimolar amount of compound (LXVII) (105 mg, 1 equiv) and compound (LXIX) (122 mg, 1 equiv) were dissolved in NMP overnight and the mixture left at room temperature. The resulting peptide resin was then isolated by washing in a fine pore sinter with 5 mL NMP, the resin was then washed with excess diethyl ether. The peptide was deprotected using the standard protocal (vide supra) for 2 hours, and isolated by precipitation with diethyl ether (50 mL). 5 mg of product (compound (LXX)) was isolated (m/z 1243.6485; calculated for C₅₅H₈₉N₁₇O₁₄S 1243.6)

### Example 12

### Synthesis of (S)—S-(2-(2,4-dimethoxyphenyl)-2-oxoethyl) 2-((tert-butoxycarbonyl)amino)-3-methylbutanethioate

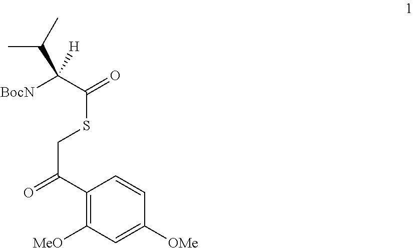

Boc-L-valine (434 mg, 2 mmol, 1 equiv) was dissolved in anhydrous tetrahydrofuran (50 mL), triethylamine (347 μL, 2 mmol, 1 equiv) was then added, and the solution was stirred for 30 minutes at 0° C. under nitrogen. Ethyl chloroformate was then added (238 μL 2 mmol, 1 equiv) and the solution was stirred for 10 minutes. Sodium hydrosulfide hydrate was then added (280 mg, 5 mmol, 2.5 equiv), and the solution was stirred for a further 2 hours at 0° C. under nitrogen. 2-Bromo-2′,4′-dimeoxthyacetophenone (518 g, 2 mmol, 1 equiv) was finally dded and the solution was stirred at room temperature under nitrogen for another 18 hours. The reaction was then quenched with methanol (0.5 mL) and concentrated in vacuo. The resulting yellow solid was then dissolved in chloroform (50 mL) to which water (100 mL) was then added. This mixture was then extracted with chloroform (2×50 mL), dried (Mg₂SO₄), filtered, and concentrated in vacuo to yield an orange oil (1000 mg.) δₕ (400 MHz CDCl₃) 7.85 (1H, d, J 9 Hz, Ph), 6.54 (1H, d, J 9 Hz, Ph), 6.46 (1H, d, J 2 Hz, Ph), 5.63-5.57 (0.3H, d br, J 8 Hz NH), 4.95-5.05 (0.7H, d br, J 8 Hz, NH), 4.33-4.43 (2H, m, SCH₂) 4.10-4.20 (1H, m, NCH), 3.95 (3H, s, OCH₃), 3.85 (3H, s, OCH₃), 2.25-2.35 (1H, m, CH(CH₃)₂), 1.46+1.44 (9H, 2×S, t-butyl) 1.00 (3H, d, J 7 Hz, CH(CH₃)₂×1), 0.89 (3H, d, J 7 Hz, CH(CH₃)₂).

### Example 13

### Synthesis of (S)-5-(2,4-dimethoxyphenyl)-3-isopropyl-3,4-dihydro-2H-1,4-thiazin-2-one and (S)-5-(2,4-dimethoxyphenyl)-3-isopropyl-3,6-dihydro-2H-1,4-thiazin-2-one

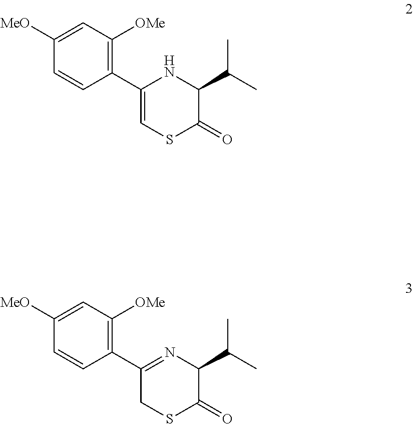 [SMILES: COc1ccc(C2=CSC(=O)[C@H](C(C)C)N2)c(OC)c1 ; COc1ccc(C2=N[C@@H](C(C)C)C(=O)SC2)c(OC)c1]

### (S)—S-(2-(2,4-dimethoxyphenyl)-2-oxoethyl) 2-((tert-butoxycarbonyl)amino)-3-methylbutanethioate (1 g, 3 mmol) was dissolved in dichloromethane (anhydrous 50 mL), trifluoroacetic acid (10 mL) was then added and the solution was stirred for 1 hour under nitrogen at room temperature. The resulting product was then concentrated in vacuo and re-dissolved in dichloromethane (50 mL) to which potassium carbonate (3 g, 21 mmol) was then added along with molecular sieves (4 Å, 200 mg). The solution was then stirred for 72 hours at room temperature. The product was obtained by vacuum filtration through Celite® followed by washing with dichloromethane (3×25 mL), the solution obtained was then dried (MgSO₄). filtered and concentrated in vacuo to yield a yellow oil (300 mg) δₕ (400 MHz CDCl₃) 7.52 (1H, d, J 9 Hz, Ph, imine) 7.27 (1H, d J 9 Hz, Ph, enamine) 6.55 (2H, d, J 9 Hz, Ph) 6.48 (1H, d, J 3 Hz, Ph, imine), 6.42 (1H, d, J 3 Hz, Ph) 5.12 (1H, d, J 3 Hz, PhC═CH), 4.49-4.45 (1H, m, NCH, imine), 4.37-4.25 (2H, m, CH₂S, imine), 3.87 (1H, s, OCH₃), 3.83 (1H, s, OCH₃), 3.39-3.37 (1H, m, NCH, enamine) 2.59-2.54 (2H, m, CH(CH₃)₂), 1.30 (3H, d, J 8 Hz, CH(CH₃)₂, enamine), 1.12 (3H, d, J 8, CH(CH₃)₂, imine).

### Example 14

### Synthesis of (3S,5R)-5-(2,4-dimethoxyphenyl)-3-isopropylthiomorpholin-2-one

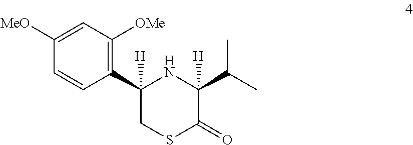 [SMILES: COc1ccc([C@@H]2CSC(=O)[C@H](C(C)C)N2)c(OC)c1]

(S)-5-(2,4-dimethoxyphenyl)-3-isopropyl-3,4-dihydro-2H-1,4-thiazin-2-one and (S)-5-(2,4-dimethoxyphenyl)-3-isopropyl-3,6-dihydro-2H-1,4-thiazin-2-one (300 mg, 1 mmol) was dissolved in anhydrous tetrahydrofuran (15 mL), sodium cyanoborohydride (164 mg, 2 equiv, 2.6 mmol) and acetic acid (0.15 mL, 2.7 mmol) were then added. The solution was stirred for 7 days hours under nitrogen at room temperature, an extraction was then performed with water (25 mL with sodium hydrogen carbonate), and ether (3×25 mL). The product was then dried (MgSO₄), filtered and concentrated in vacuo to give a yellow oil (20 mg) δₕ (400 MHz CDCl₃) 7.35 (1H, d, J 8 Hz, Ph), 6.48 (2H, m, Ph), 4.49 (1H, dd, J 11 Hz J¹ 3 Hz CH₂S, ×1), 3.85 (3H, s, OCH₃), 3.80 (3H, s, OCH₃) 3.67 (1H, d, J 3 Hz, NCH) 3.47-3.42 (1H, t, J 11 Hz, PhCH), 3.12 (1H, dd, J 11 Hz, J¹ 3 Hz, CH₂S, ×1) 2.53 (1H, dsept, J 7 Hz, J 3 Hz, CHCH₃), 1.75-1.95 (1H, br, NH) 1.00 (6H, d, J 7 Hz, CHCH₃). δ_{c} (100 MHz CDCl₃) 16.8, 19.3, 30.2, 36.6, 52.4, 55.4, 74.0, 98.7, 104.4, 123.0, 126.9, 157.4, 160.6, 201.6.

### Example 15

### Synthesis of (S)—S-(2-(2,4-dimethoxyphenyl)-2-oxoethyl) 2((tertbutoxycarbonyl)amino)propanethioate

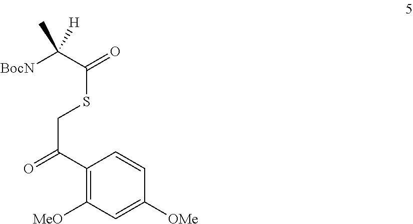

To anhydrous tetrahydrofuran (50 mL) Boc-L-alanine (378 mg, 2 mmol, 1 equiv.) was added along with triethylamine (347 μl) and the reaction was stirred for 30 minutes at 0° under nitrogen. Ethyl chloroformate (238 μl) was then added followed by stirring for 10 minutes, and sodium hydrosulfide hydrate (280 mg, 5 mmol, 2.5 equiv.) was subsequently added. The reaction was then stirred for 2 hours at 0° C. under nitrogen when 2-bromo-2′,4′-dimeoxthyacetophenone (518 mg, 2 mmol, 1 equiv.) was added and the reaction was stirred for 18 hours under nitrogen at room temperature. The reaction was quenched with methanol (0.5 mL) followed by concentration in vacuo, the resulting residue was dissolved in chloroform (50 mL) and washed with water (100 mL), followed by extraction of the aqueous phase with chloroform (2×50 ml). The product was dried (Mg₂SO₄), filtered, and concentrated in vacuo to yield a yellow oil (800 mg). δₕ (400 MHz CDCl₃) 7.86 (1H, d, J 9 Hz, Ph), 6.55 (1H, d, J 9 Hz, Ph), 6.46 (1H, d, J 2.5 Hz, Ph), 4.99 (1H, d, J 8 Hz, NH), 4.60 (2H, m, SCH₂) 3.92 (3H, s, OCH₃) 3.86 (3H, s, OCH₃) 3.02-3.10 (1H, m, CHCH₃) 1.44 (9H, s, t-butyl) 1.40 (3H, d, J 7, CHCH₃)

### Example 16

### Synthesis of (S)-5-(2,4-dimethoxyphenyl)-3-methyl-3,4-dihydro-2H-1,4-thiazin-2-one and (S)-5-(2,4-dimethoxyphenyl)-3-methyl-3,6-dihydro-2H-1,4-thiazin-2-one

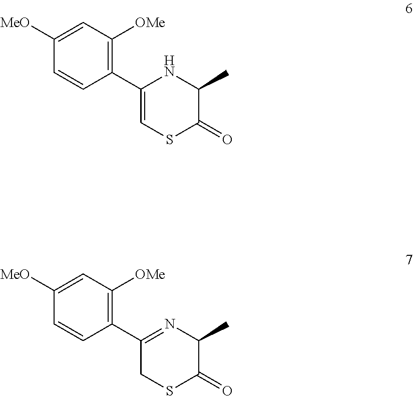 [SMILES: COc1ccc(C2=CSC(=O)[C@H](C)N2)c(OC)c1 ; COc1ccc(C2=N[C@@H](C)C(=O)SC2)c(OC)c1]

(S)—S-(2-(2,4-dimethoxyphenyl)-2-oxoethyl) 2((tertbutoxycarbonyl)amino) propanethioate (800 mg, 2 mmol) was dissolved in dichloromethane (20 mL), trifluoroacetic acid (4 mL) was then added and the mixture was stirred for 1 hour under nitrogen at room temperature. The product was then concentrated in vacuo, and redissolved in dichloromethane (50 mL) to which potassium carbonate (2.05 g 15 mmol) was added, along with 4 Å molecular sieves (200 mg). The solution was then stirred for 72 hours at room temperature. The product was filtered through Celite®, and the Celite® washed with dichloromethane (2×50 mL) The resulting extracted was dried (MgSO₄), filtered and concentrated in vacuo to yield a yellow oil 200 mg. δₕ (400 MHz CDCl₃) 7.48 (1H, d, J 9 Hz, Ph), 6.53 (1H, d, J 9 Hz, Ph) 6.49 (1H, d, J 3 Hz, Ph) 5.11 (1H, d, J 3 Hz, PhC═CH), 4.34 (1H, m, CH₂S, ×1), 4.16 (1H, m, CH₂S, ×1); 3.87 (3H, s, OCH₃), 3.84 (3H, s, OCH₃), 3.49-3.51 (1H, m, CHCH₃), 1.68 (3H, d, CHCH₃ enamine), 1.25 (3H, s, CHCH₃ imine).

### Example 17

### Synthesis of (3S,5R)-5-(2,4-dimethoxyphenyl)-3-methylthiomorpholin-2-one and (3S,5S)-5-(2,4-dimethoxyphenyl)-3-methylthiomorpholin-2-one

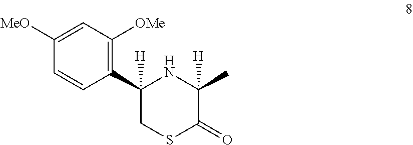 [SMILES: COc1ccc([C@@H]2CSC(=O)[C@H](C)N2)c(OC)c1]

(S)-5-(2,4-dimethoxyphenyl)-3-methyl-3,4-dihydro-2H-1,4-thiazin-2-one and (S)-5-(2,4-dimethoxyphenyl)-3-methyl-3,6-dihydro-2H-1,4-thiazin-2-one (200 mg, 0.75 mmol) was dissolved in anhydrous tetrahydrofuran (15 ml). Sodium cyanoborohydride (164 mg, 2.6 mmol) was then added along with acetic acid (0.15 mL, 2.7 mmol), the mixture was then stirred under nitrogen for 7 days at room temperature. The mixture was then diluted with ether (25 mL×3) and washed water (30 mL with 25 mg sodium hydrogen carbonate). The extract was dried (MgSO₄), filtered, and concentrated in vacuo, to yield a yellow oil. δₕ (400 MHz CDCl₃) 7.26 (1H, d, J 5 Hz, Ph), 6.54 (1H, d, J 8 Hz, Ph), 6.50 (1H, d, J 2 Hz, Ph), 4.43 (1H, dd, J=11 Hz, J¹ 3 Hz, SCH₂), 3.86 (3H, s, OCH₃), 3.81 (3H, s, OCH₃), 3.64-3.59 (1H, s, NCH), 3.43 (1H, t, J 13 Hz, PhCH), 2.55 (1H, dd, J 13 Hz, J¹ 3 Hz, SCH₂×1), 1.29 (3H, d, J 3 Hz CHCH₃).

### Example 18

### Synthesis of (S)—S-(2-(2,4-dimethoxyphenyl)-2-oxoethyl) 2-((tert-butoxycarbonyl)amino)-3-phenylpropanethioate

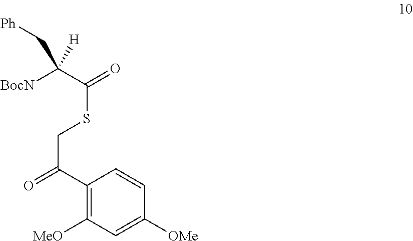

To anhydrous tetrahydrofuran (50 mL) Boc-L-phenylalanine (530 mg, 2 mmol, 1 equiv.) and triethylamine (347 μl) were added and the mixture was stirred for 30 minutes at 0° C. under nitrogen. Ethyl chloroformate (238 μl) was then added, was followed by stirring for 10 minutes and subsequent addition of sodium hydrogen sulfide (280 mg, 5 mmol, 5 equiv) The solution was stirred for 2 hours at 0° C. under nitrogen and, to this 2-bromo-2′,4′-dimeoxthyacetophenone (518 mg, 2 mmol, 1 equiv.) was then added, and the mixture stirred for a further 18 hours at room temperature under nitrogen. The reaction was then quenched with methanol (0.5 mL) followed by concentration in vacuo. The residue was redissolved in chloroform (50 mL) washed with water (100 mL) and the aqueous phase extracted with chloroform (2×50 mL). The solution was then dried (MgSO₄), filtered, and concentrated in vacuo to yield a yellow oil. δₕ (400 MHz CDCl₃), 7.87 (1H, d, J 9 Hz, Ph), 7.36-7.16 (5H, m, Ph), 6.55 (1H, d, J 9 Hz, Ph), 6.46 (1H, d, J 3 Hz, Ph), 4.91-4.89 (1H, d, J 8 Hz, NH), 4.69-4.63 (1H, m, NCH), 4.47-4.43 (1H, d, J 16 Hz, CH₂S×1), 4.35-4.34 (1H, d, J 4 Hz, CH₂S×1), 3.92 (3H, s, OCH₃), 3.87 (3H, s, OCH₃), 3.16-3.01 (2H, m, CHCH₂Ph), 1.41-1.39 (9H, d, J 8 Hz, t-butyl).

### Example 19

### Synthesis of (S)-3-benzyl-5-(2,4-dimethoxyphenyl)-3,4-dihydro-2H-1,4-thiazin-2-one and (S)-3-benzyl-5-(2,4-dimethoxyphenyl)-3,6-dihydro-2H-1,4-thiazin-2-one

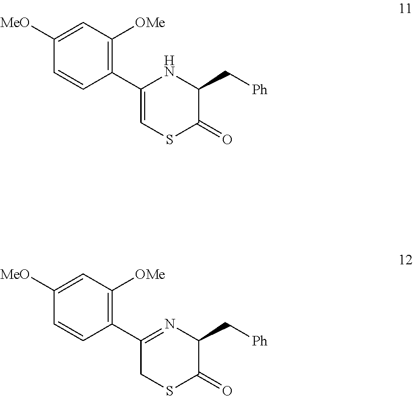 [SMILES: COc1ccc(C2=CSC(=O)[C@H](Cc3ccccc3)N2)c(OC)c1 ; COc1ccc(C2=N[C@@H](Cc3ccccc3)C(=O)SC2)c(OC)c1]

(S)—S-(2-(2,4-dimethoxyphenyl)-2-oxoethyl) 2-((tert-butoxycarbonyl)amino)-3-phenylpropanethioate (1 g, 2.2 mmol) was dissolved in anhydrous dicholoromethane (20 mL) to which trifluoroacetic acid (4 mL) was added. The solution was then stirred for 1 hour at room temperature under nitrogen, concentrated in vacuo and the residue redissolved in dichloromethane (50 mL) and potassium carbonate (2 g, 14.5 mmol) and 4 Å molecular sieves (200 mg) were added. The mixture was then stirred under nitrogen at room temperature for 72 hours then filtered through Celite® followed by washing the Celite® with dichloromethane (2×50 mL) The extract was then dried (MgSO₄), filtered and concentrated in vacuo to yield a yellow oil. δₕ (400 MHz CDCl₃) 7.47 (1H, d, J 9 Hz, Ph, imine), 7.39-7.15 (5H, m, Ph), 7.16 (1H, d, J 9 Hz, enamine), 6.53 (1H, d, J 9 Hz, Ph, imine), 6.47 (1H, d, J 3, Ph, imine) 6.41 (1H, d, J 9 Hz, Ph, enamine) 6.30 (1H, d, J 3 Hz, Ph enamine), 5.18 (1H, d, J 3 Hz, PhC═CH), 4.45-4.51 (1H, br, NH), 4.37-4.17 (1H, m, SCH₂, ×1) 3.99-3.92 (1H, m, SCH₂, ×1), 3.87 (3H, s, OCH₃), 3.84 (3H, s, OCH₃), 3.76 (1H, s, NCH, imine), 3.27 (1H, s, NCH, enamine) 3.59-3.50 (2H, m, CH₂Ph, imine), 2.81-2.75 (2H, m, CH₂Ph, enamine).

### Example 20

### Synthesis of (3S,5R)-3-benzyl-5-(2,4-dimethoxyphenyl)thiomorpholin-2-one

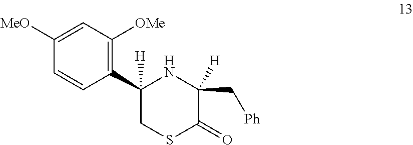 [SMILES: COc1ccc([C@@H]2CSC(=O)[C@H](Cc3ccccc3)N2)c(OC)c1]

To a solution of (S)-3-benzyl-5-(2,4-dimethoxyphenyl)-3,4-dihydro-2H-1,4-thiazin-2-one and (S)-3-benzyl-5-(2,4-dimethoxyphenyl)-3,6-dihydro-2H-1,4-thiazin-2-one (751 mg, 2.2 mmol) in anhydrous tetrahydrofuran (30 mL), sodium cyanoborohydride (250 mg, 4 mmol) and acetic acid (250 μl, 2.40 mmol) were added. The mixture was then stirred for 7 days under nitrogen at room temperature. The mixture was then extracted with ether (3×25 mL) and washed with water (50 mL containing sodium hydrogen carbonate 25 mg). The solution was then dried (MgSO₄), filtered and concentrated in vacuo. δₕ (400 MHz CDCl₃) 7.37 (1H, d, J 7 Hz, Ph), 7.32-7.20 (5H, m, Ph), 6.46 (1H, d, J 7 Hz, Ph), 6.45 (1H, d, J 3 Hz, Ph), 4.40 (1H, dd, J 11 Hz, J¹ 3 Hz, CH₂S, ×1), 3.96-3.90 (1H, m, CHCH₂Ph), 3.75 (3H, s, OCH₃), 3.67 (3H, s, OCH₃), 3.47-3.41 (1H, m, PhCH), 3.10 (1H, dd, J 11 Hz, J¹ 3 Hz, CH₂S, ×1), 2.95-2.90 (2H, m, CHCH₂Ph). δ_{c} (100 MHz CDCl₃) 25.6, 36.4, 37.5, 53.0, 55.2, 55.4, 68.0, 69.8, 98.7, 104.4, 122.4, 126.7, 126.9, 128.7, 129.4, 137.8, 157.4, 160.8, 200.1.

### Example 21

### Synthesis of (S)—S-(2-(2,4-dimethoxyphenyl)-2-oxoethyl) 2-((tert-butoxycarbonyl)amino)-3-(4-hydroxyphenyl)propanethioate

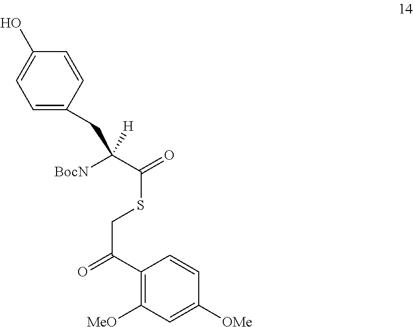

To anhydrous tetrahydrofuran (50 mL) Boc-L-tyrosine (562 mg, 2 mmol, 1 equiv.) triethylamine (347 μl) were added and the reaction was stirred for 30 minutes at 0° C. under nitrogen. Ethyl chloroformate (238 μl) was then added followed by stirring for 10 minutes, then sodium hydrogen sulfide hydrate (280 mg, 5 mmol, 5 equiv.) was added and the mixture was stirred for 2 hours at 0° C. under nitrogen. 2-Bromo-2′,4′-dimeoxthyacetophenone (518 mg, 2 mmol, 1 equiv.) was added and the mixture was stirred for 18 hours at room temperature under nitrogen. The reaction was then quenched with methanol (0.5 mL) and concentrated in vacuo. The residue was redissolved in chloroform (50 mL), followed by washed with water (100 mL) and extracting the aqueous washings with chloroform (2×50 mL). The organic extracts were then dried (MgSO₄), filtered and concentrated in vacuo to yield a yellow oil (1.22 g). δₕ (400 MHz CDCl₃) 7.85 (1H, d, J 9 Hz, Ph), 7.19 (2H, d, J 9 Hz, Ph) 7.10 (2H, d, J 9 Hz, Ph), 6.55 (1H, d, J 9 Hz, Ph), 6.46 (1H, d, J 3 Hz, Ph), 5.03 (1H, d, J 9 Hz, NH), 4.66-4.54 (1H, m, NCH×1), 4.45-4.25 (2H, m, SCH₂) 3.91 (3H, s, OCH₃), 3.87 (3H, s, OCH₃), 3.21-3.16 (1H, m, CHCH₂Ph×1), 3.08-3.00 (1H, m, CHCH₂Ph×1), 1.40-1.37 (9H, m, t-butyl). δ_{c} (100 MHz CDCl₃) 14.6, 25.7, 28.5, 55.5, 65.0, 68.0, 98.3, 105.7, 121.1, 130.4, 133.6, 165.3, 200.5.

### Example 22

### Synthesis of (S)-5-(2,4-dimethoxyphenyl)-3-(4-hydroxybenzyl)-3,4-dihydro-2H-1,4-thiazin-2-one and (S)-5-(2,4-dimethoxyphenyl)-3-(4-hydroxybenzyl)-3,6-dihydro-2H-1,4-thiazin-2-one

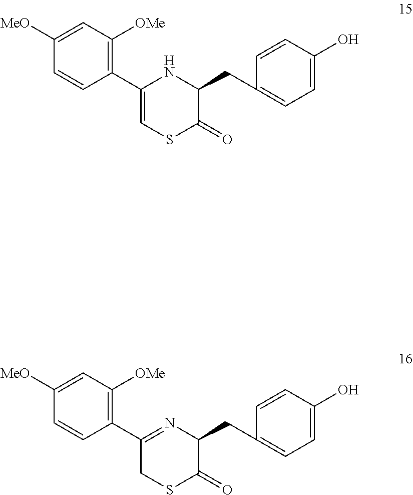 [SMILES: COc1ccc(C2=CSC(=O)[C@H](Cc3ccc(O)cc3)N2)c(OC)c1 ; COc1ccc(C2=N[C@@H](Cc3ccc(O)cc3)C(=O)SC2)c(OC)c1]

To a solution of (S)—S-(2-(2,4-dimethoxyphenyl)-2-oxoethyl) 2-((tert-butoxycarbonyl)amino)-3-(4-hydroxyphenyl)propanethioate (1.22 g, 2.5 mmol) in anhydrous dichloromethane (30 mL), trifluoroacetic acid (6 mL) was added. The solution was then stirred at room temperature under nitrogen for 1 hour. The mixture was then concentrated in vacuo and redissolved in anhydrous dichloromethane (50 mL) to which potassium carbonate (1.5 g, 10 mmol) was added, along with 4 Å molecular sieves (200 mg). The solution was stirred at room temperature under nitrogen for 72 hours, then filtered through Celite®, followed by washing the Celite® with dichloromethane (2×50 mL) The solution was dried (MgSO₄), and concentrated in vacuo, to yield a yellow oil. δₕ (400 MHz CDCl₃) 7.85 (1H, d, J 9 Hz, Ph, enamine) 7.45 (1H, d, J 9 Hz, Ph, imine), 7.38 (2H, d, J 9 Hz, Ph), 7.08 (2H, d, J 9 Hz, Ph), 6.54 (1H, d, J 9 Hz, Ph, imine), 6.46 (1H, d, J 3 Hz, Ph, imine), 6.43 (1H, d, J 9 Hz, Ph, enamine), 6.32 (1H, d, J 3 Hz, Ph, enamine), 5.18 (1H, PhC═CH), 4.37-4.22 (1H, m, SCH₂, imine×1) 3.95-3.90 (1H, d, SCH₂, imine×1) 3.83 (3H, s, OCH₃), 3.80 (3H, s, OCH₃), 3.78 (1H, s, NCH, imine), 3.32 (1H, NCH, enamine), 3.57-3.52 (1H, dd, J 20 Hz, CHCH₂Ph, ×1), 3.55-3.55 (2H, m, CH₂Ph, imine) 3.30-3.25 (2H, m, CH₂Ph, enamine)

### Example 23

### Synthesis of (3S,5R)-5-(2,4-dimethoxyphenyl)-3-(4-hydroxybenzyl)thiomorpholin-2-one

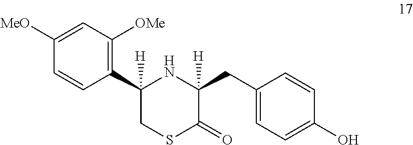 [SMILES: COc1ccc([C@@H]2CSC(=O)[C@H](Cc3ccc(O)cc3)N2)c(OC)c1]

To a solution of (S)-5-(2,4-dimethoxyphenyl)-3-(4-hydroxybenzyl)-3,4-dihydro-2H-1,4-thiazin-2-one and (S)-5-(2,4-dimethoxyphenyl)-3-(4-hydroxybenzyl)-3,6-dihydro-2H-1,4-thiazin-2-one (130 mg, 0.36 mmol), in anhydrous tetrahydrofuran (15 mL) sodium cyanoborohydride (100 mg, 1.5 mmol) and acetic acid (100 μl) were added. The reaction was then stirred for 7 days at room temperature under nitrogen. The mixture was then extracted with ether (3×25 mL) washed with water (50 mL with sodium hydrogen carbonate), the product was then dried (MgSO₄), filtered and concentrated in vacuo. δₕ (400 MHz CDCl₃) 7.26 (2H, d, J 9 Hz, Ph), 7.22 (1H, d, J 9 Hz, Ph), 7.12 (2H, d, J 9 Hz, Ph), 6.46 (1H, d, J 9 Hz, Ph), 6.39 (1H, d, J 3 Hz, Ph), 4.32 (1H, dd, J 11 Hz, J′ 3 Hz SCH₂×1), 4.20-4.30 (2H, m, CHCH₂Ph), 3.90-3.80 (1H, t, J 6 Hz, J 1 Hz, PhCH) 3.79 (3H, s, OCH₃), 3.70 (1H, s, NCH) 3.67 (3H, s, OCH₃) 3.10 (1H, dd, J 11 Hz, J′ 3 Hz SCH₂×1)

### Example 24

### Synthesis of (S)—S-(2-(2,4-dimethoxyphenyl)-2-oxoethyl) 2-((tert-butoxycarbonyl)amino)-3-(1H-indol-3-yl)propanethioate

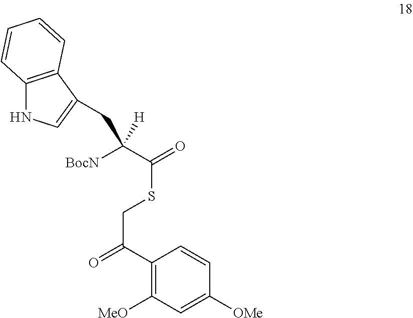

To anhydrous tetrahydrofuran (50 mL) Boc-tryptophan (608 mg, 2 mmol, 1 equiv.) was added along with triethylamine (347 μl, 1 equiv.), the solution was then stirred at 0° C. for 30 minutes under nitrogen. Ethyl chloroformate (238 μl, 1 equiv.) was then added which was followed by stirring for 10 minutes. Sodium hydrosulfide (280 mg, 5 mmol, 5 equiv.) was then added, and the solution was stirred for 2 hours at 0° C. under nitrogen. 2-Bromo-2′,4′-dimethoxyacetophenone (518 mg, 2 mmol, 1 equiv.) was added and the reaction was stirred for further 18 hours under nitrogen at room temperature. The reaction was quenched with methanol (0.5 mL) and the solution was concentrated in vacuo. The residue was then dissolved in chloroform (50 mL) and extracted with water (100 mL) and chloroform (2×50 mL). The resulting solution was dried (MgSO₄), filtered, and concentrated in vacuo to give a yellow oil (877 mg). δₕ (400 MHz CDCl₃) 8.76-8.75 (1H, br s, ═C—CNH, indole) 7.85 (1H, d, J 9 Hz, Ph), 7.54 (1H, d, J 9 Hz, Ph, indole), 7.32 (1H, d, J 9 Hz, Ph, indole), 7.16-7.12 (1H, m, Ph, indole), 7.09-7.07 (1H, m, Ph, indole), 6.51 (1H, d, J 9 Hz, Ph), 6.43 (1H, d, J 3 Hz, Ph), 5.16 (1H, d, J 9 Hz, NH), 5.27 (1H, m, NCH), 4.44-4.31 (2H, m, SCH₂) 3.86 (3H, s, OCH₃), 3.82 (3H, s, OCH₃), 2.90 (2H, q, J 14 Hz, J′ 8 Hz, CHCH₂Ph).

### Example 25

### Synthesis of (S)-3-((1H-indol-3-yl)methyl)-5-(2,4-dimethoxyphenyl)-3,4-dihydro-2H-1,4-thiazin-2-one and (S)-3-((1H-indol-3-yl)methyl)-5-(2,4-dimethoxyphenyl)-3,6-dihydro-2H-1,4-thiazin-2-one

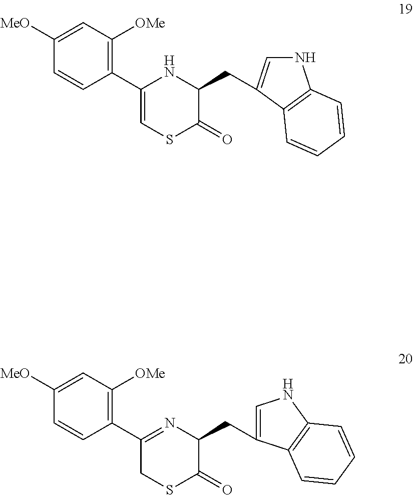 [SMILES: COc1ccc(C2=CSC(=O)[C@H](Cc3c[nH]c4ccccc34)N2)c(OC)c1 ; COc1ccc(C2=N[C@@H](Cc3c[nH]c4ccccc34)C(=O)SC2)c(OC)c1]

(S)—S-(2-(2,4-dimethoxyphenyl)-2-oxoethyl) 2-((tert-butoxycarbonyl)amino)-3-(1H-indol-3-yl)propanethioate (877 mg, 1.8 mmol) was dissolved in dicholoromethane (50 mL) along with trifluoroacetic acid (10 mL). The solution was then stirred for 1 hour at room temperature. The product was then concentrated in vacuo and redissolved in dichloromethane (100 mL). Potassium carbonate (2 g, 10 mmol) and 4 Å molecular sieves (200 mg) were then added and the solution was stirred for 72 hours at room temperature under nitrogen. The product was then filtered through Celite®, followed by washing the Celite® with dichloromethane (2×50 mL) the filtrate was then dried with (MgSO₄), which was subsequently filtered and concentrated in vacuo to give a yellow oil (567 mg). δₕ (400 MHz CDCl₃)

### Example 27

### Synthesis of (3S)-3-((1H-indol-3-yl)methyl)-5-(2,4-dimethoxyphenyl)thiomorpholin-2-one

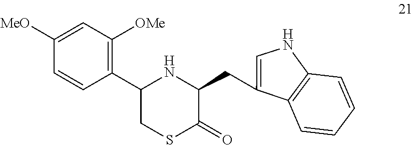 [SMILES: COc1ccc(C2CSC(=O)[C@H](Cc3c[nH]c4ccccc34)N2)c(OC)c1]

(S)-3-((1H-indol-3-yl)methyl)-5-(2,4-dimethoxyphenyl)-3,4-dihydro-2H-1,4-thiazin-2-one and (S)-3-((1H-indol-3-yl)methyl)-5-(2,4-dimethoxyphenyl)-3,6-dihydro-2H-1,4-thiazin-2-one (567 mg, 1.5 mmol) were dissolved in anhydrous tetrahydrofuran (15 mL). To this solution sodium cyanoborohydride (300 mg, 4.8 mmol) and acetic acid (300 μl) were added and the solution was stirred for 7 days under nitrogen at room temperature. The mixture was washed with water (50 mL with sodium hydrogen carbonate 25 mg) and the aqueous phase extracted with ether (3×50 mL). The resulting solution was dried (MgSO₄), filtered and concentrated in vacuo to give a yellow oil (370 mg). HRMS for C₂₁H₂₂N₂O₃S requires 382.1351 found 385.2 (unreduced dehydrothiaziones) and 386.2 (reduced thiomorpholinone product) Once again the proton NMR spectra was difficult to interpret as it was so complex. However double doublet at 4.35 and 2.60 ppm correspond to the two SCH₂ protons. These signals along with the presence of the 7.40-7.00 ppm aromatic and 3.72 and 3.70 ppm methoxy confirm that reduced material is present.

### Example 28

### Synthesis of S-(2-(2,4-dimethoxyphenol)-2-oxoethyl) 2-((tert-butoxycarbonyl)amino)ethanethioate

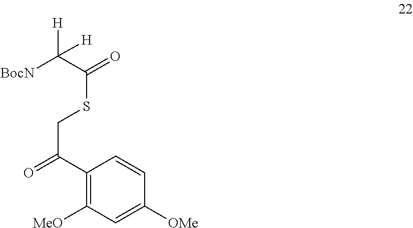

To anhydrous tetrahydrofuran (50 mL) Boc-glycine (375 mg, 2 mmol, 1 equiv.) was added, followed by the addition of triethylamine (347 μL) after which the reaction was stirred for 30 minutes at 0° C. under nitrogen. Ethyl chloroformate (238 μL) was added and the reaction was stirred for 10 minutes, sodium hydrosulfide (280 mg) was then added and the reaction was stirred for 2 hours at 0° C. under nitrogen. 2-Bromo-2′,4′-dimeoxthyacetophenone (518 mg, 2 mmol, 1 equiv.) was added, after which the reaction was stirred for 18 hours at room temperature under nitrogen. The reaction was quenched with methanol (0.5 mL) and concentrated in vacuo. The residue was then redissolved in chloroform (50 mL) which was washed with water (100 mL) and the aqueous phase extracted with chloroform (2×50 mL). The organic extracts were then dried (MgSO₄), filtered, and concentrated in vacuo to give a yellow oil (376 mg). δₕ (400 MHz CDCl₃) 7.85 (1H, d, J 9 Hz, Ph), 6.55 (1H, d, J 9 Hz, Ph), 6.46 (1H, d, J 3 Hz, Ph). 5.32 (1H, b, NH), 4.42 (2H, s, NHCH₂), 4.08 (2H, d, J 6 Hz, SCH₂), 3.92 (3H, s, OCH₃), 3.74 (3H, s, OCH₃), 1.46 (9H, s, t-butyl).

### Example 29

### Synthesis of 5-(2,4-dimethoxyphenyl)-3,4-dihydro-2H-1,4-thiazin-2-one and 5-(2,4-dimethoxyphenyl)-3,6-dihydro-2H-1,4-thiazin-2-one

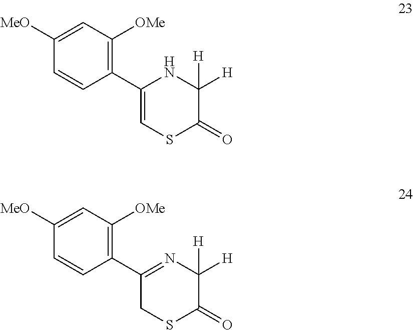 [SMILES: COc1ccc(C2=CSC(=O)CN2)c(OC)c1 ; COc1ccc(C2=NCC(=O)SC2)c(OC)c1]

S-(2-(2,4-dimethoxyphenyl)-2-oxoethyl)-2-((tert-butoxycarbonyl)amino) ethanethioate (376 mg, 1 mmol) was dissolved in dichloromethane (20 mL) to which trifluoroacetic acid (4 mL) was added and the solution was then stirred at room temperature for 1 hour. The mixture was then concentrated in vacuo followed by redissolving the residue in dichloromethane (50 mL) and potassium carbonate (1 g, 7 mmol) and 4 Å molecular sieves (200 mg) were then added. The solution was then stirred at room temperature under nitrogen for 48 hours, then filtered through Celite® followed by washing the Celite® with dichloromethane (2×50 mL), the filtrate was then dried (MgSO₄), filtered and concentrated in vacuo to give a yellow oil (mg). δₕ (400 MHz CDCl₃) 7.89 (1H, d, J 8, Ph, enamine) 7.51 (1H, d, J 8 Hz, Ph, imine), 6.53 (2H, d, J 8 Hz, Ph, imine and enamine), 6.49 (1H, d, J 2 Hz, Ph, imine), 6.46 (1H, d, J 2 Hz, Ph, enamine), 5.21 (1H, d, J 2 Hz, PhC═CH), 4.51 (1H, s, NHCH₂×1 imine), 4.37 (1H, s, NHCH₂×1 enamine) 4.26 (2H, q, J 14 Hz, J′ 7 Hz, SCH₂), 4.13 (1H, s, NHCH₂×1, imine), 3.92 (1H, s, NHCH₂×1 enamine) 3.87 (3H, s, OCH₃), 3.85 (3H, s, OCH₃)

These together with other objects of the invention, along with the various features of novelty that characterize the invention, are pointed out with particularity in the claims annexed to and forming a part of this disclosure.

1. A process for the preparation of a compound of formula (V) 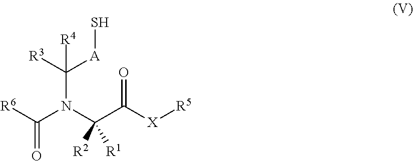
or a salt form thereof,
wherein R¹ to R⁶ are independently selected substituents;
A is selected from a bond and (CR⁷R⁸)ₙ wherein each of R⁷ and R⁸ is independently selected from the group consisting of H, C₁-C₆ alkyl optionally substituted with from one to five groups independently selected from hydroxy, C₁-C₃ alkoxy, and cyano; C₁-C₆ alkoxycarbonyl, C₁-C₆ haloalkyl, C₆-C₁₀ aryl optionally substituted with from one to five groups independently selected from hydroxy, C₁-C₃ alkoxy, halogen, nitro and cyano; or, taken together with the carbon atom to which they are attached, R⁷ and R⁸ form a C₃-C₇ cycloalkyl ring;
n is 1 or 2;
X is selected from the group consisting of O, S and NR⁹, wherein R⁹ is selected from H and C₁-C₆ alkyl and C₆-C₁₀ aryl
i) reacting a compound of formula (VI) 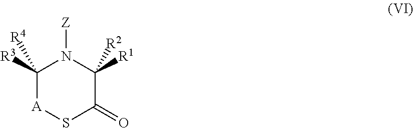
wherein R¹ to R⁴ and A are as defined above and Z is selected from the group consisting of H, benzyl, benzyloxycarbonyl, t-butyloxycarbonyl (BOC), 9H-fluoren-9-ylmethoxycarbonyl (FMOC), allyloxycarbonyl (alloc), and Si((C₁-C₁₀)alkyl)₃, with a compound of formula (VII) 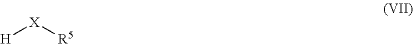
or a reactive derivative thereof, wherein R⁵ and X are as defined above to give a compound of formula (VIII) 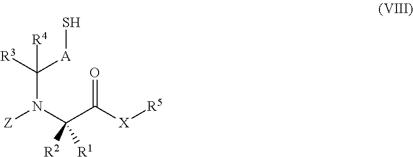
wherein R¹ to R⁵, A and Z are as defined above;
ii) optionally deprotecting compound of formula (VIII) wherein Z is a protecting group to give a compound of formula (VIII) wherein Z is H,
iii) reacting the compound of formula (VIII) wherein Z is H with an acylating agent of formula (IX) 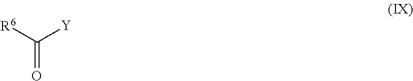
wherein R⁶ is an optionally protected peptide optionally attached to a solid support, optionally via a linker; and Y is a leaving group to give a compound of formula (V).

2. A process according to claim 1 wherein compound (IX) is a thioester.

3. A process according to claim 1 wherein compound of formula (IX) has the formula (XIII) 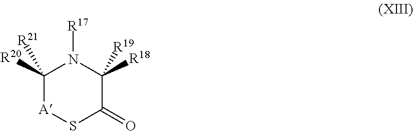
wherein R¹⁷ to R²¹ are independently selected substituents; and
A′ is selected from a bond, and (CR³⁷R³⁸)ₙ wherein each of R³⁷ and R³⁸ is independently selected from the group consisting of H, C₁-C₆ alkyl optionally substituted with from one to three groups independently selected from hydroxy, C₁-C₃ alkoxy, and cyano; C₁-C₆ alkoxycarbonyl, and C₁-C₆ haloalkyl; or, taken together with the carbon atom to which they are attached, R³⁷ and R³⁸ form a C₃-C₇ cycloalkyl ring; and
n is 1 or 2.

4. A process according to claim 3 wherein R¹⁷ is an optionally protected peptide.

5. A process according to claim 4 wherein R¹⁷ comprises at least a 9H-fluoren-9-ylmethoxycarbonyl (FMOC) protecting group.

6. A process according to claim 1 wherein X is NH.

7. A process according to claim 1 wherein R⁴ is H.

8. A process according to claim 1 wherein R² is H.

9. A process according to claim 1 wherein A is CH₂.

10. A process according to claim 1 wherein R³ is aryl, optionally attached to a solid support, optionally via a linker.

11. A process according to claim 1 wherein R⁵ is an optionally protected peptide optionally attached to a solid support, optionally via a linker.

12. A process according to claim 1 wherein R⁶ is an optionally protected peptide optionally attached to a solid support, optionally via a linker.

13. A process according to claim 1 comprising the further step of converting the compound of formula (V) to a compound of formula (XXXIV) 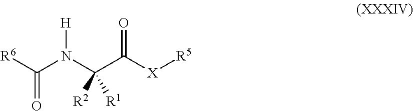
wherein R¹, R², R⁵, R⁶, and X are as defined in claim 1.

14. A process according to claim 11 comprising a further deprotection step or steps to give a free peptide.

15. A process for the preparation of a compound of formula (XLVII) 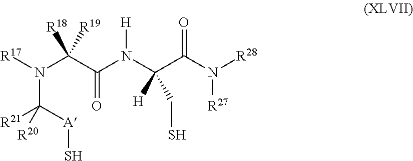
wherein R¹⁷ to R²¹ are independently selected substituents; and
A′ is selected from a bond, and (CR⁷R⁸)ₙ wherein each of R⁷ and R⁸ is independently selected from the group consisting of H, C₁-C₆ alkyl optionally substituted with from one to three groups independently selected from hydroxy, C₁-C₃ alkoxy, and cyano; C₁-C₆ alkoxycarbonyl, and C₁-C₆ haloalkyl; or, taken together with the carbon atom to which they are attached, R⁷ and R⁸ form a C₃-C₇ cycloalkyl ring;
n is 1 or 2;
R²⁸ is H or is an optionally protected peptide, optionally attached to a solid support, optionally via a linker;
R²⁷ is H or taken together with the nitrogen to which it is attached and the side chain of the adjacent amino acid forms a pyrrolidine ring; comprising
i) reacting a peptide with an N-terminal cysteine residue of formula (XLVI) 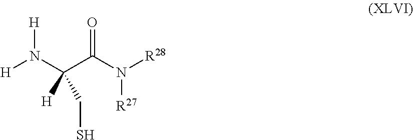
wherein R²⁷ and R²⁸ are as defined above,
with a compound of formula (XIII) 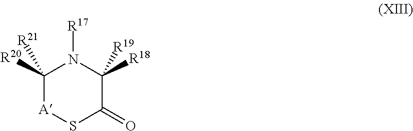
wherein R¹⁷ to R²¹ and A′ are as defined above.

16. A compound of the formula (X) 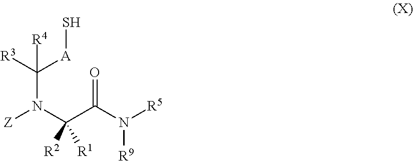
or a salt form thereof, wherein R¹ to R⁵, R⁹ A and Z are as defined in claim 1.

17. (canceled)

18. A compound of formula (VI) 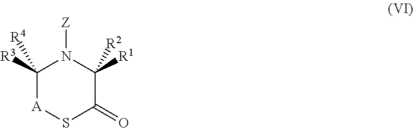
or a salt form thereof, wherein R¹ to R⁴ and A are as defined as in claim 1, and Z is a group selected from the group consisting of benzyl, benzyloxycarbonyl, t-butyloxycarbonyl (BOC), 9H-fluoren-9-ylmethoxycarbonyl (FMOC), allyloxycarbonyl (alloc), and Si((C₁-C₁₀)alkyl)₃.

19. A compound of formula (VI) according to claim 18 wherein Z is t-butyloxycarbonyl (BOC).
